(19) United States
(12) Patent Application Publication
(10) Pub. No.: US 2023/0052677 A1
(43) Pub. Date: Feb. 16, 2023

(54) MOLECULE DESIGN

(71) Applicant: Flagship Pioneering Innovations VI, LLC, Cambridge, MA (US)

(72) Inventors: Umut ESER, Lexington, MA (US); Fabian Alexander WOLF, Munich (DE); Nicholas McCartney PLUGIS, Cambridge, MA (US)

(21) Appl. No.: 17/792,639

(22) PCT Filed: Jan. 14, 2021
(86) PCT No.: PCT/US21/13451

§ 371 Date: Jul. 13, 2022

Related U.S. Application Data
(60) Provisional application No. 62/961,112, filed on Jan. 14, 2020.

(51) Int. Cl. G16B 5/00; G16B 15/30; G16B 40/20

(52) U.S. Cl. CPC G16B 5/00; G16B 15/30; G16B 40/20

(57) ABSTRACT

Systems and methods of discovering compounds with biological properties are provided. A first training dataset is obtained, including chemical structures and biological properties. Projections of compounds are obtained by projecting chemical structure information into a latent representation space using encoder weights. Compounds are classified by inputting projections into the classifier using classifier weights. The encoder and classifier are trained by comparing the classification of each compound to actual biological properties and updating the respective weights. A second training dataset is obtained including chemical structures. Projections of compounds are obtained by projecting chemical structure information into a latent representation space using encoder weights. Chemical structures are obtained by inputting projections into a decoder using decoder weights. The decoder is trained by comparing outputted and actual chemical structures and updating the respective weights. Candidate compounds not present in the first and second datasets are identified using the trained encoder, classifier, and decoder.

### CROSS REFERENCE TO RELATED APPLICATIONS

This application claims priority to United States Provisional Patent Application No. 62/961,112, entitled “Molecule Design.” filed Jan. 14, 2020, the contents of which are hereby incorporated by reference in its entirety for all purposes.

### TECHNICAL FIELD

The present disclosure relates generally to systems and methods for molecular design. More particularly, the present disclosure relates to using machine learning to discover compounds with biological properties.

### BACKGROUND

The study of cellular mechanisms and the chemical compounds and intermediates underlying such biological processes is important for understanding the etiology, manifestation, and progression of disease. Existing drug discovery methods, whether traditional high-throughput screens or methods employing in silico approaches remain inefficient and unable to meet existing medical needs.

There is a need in the art to overcome the existing challenges faced by drug discovery using improved methods for generating and optimizing drug structures to manipulate one or many target cell states (e.g., through the respective molecular signatures). In particular, there is a need in the art for improved methods for drug discovery, for example, to refine understanding of natural diverse cell states, to reveal key transition states where cells choose alternative states, to uncover the molecular drivers underlying cell state changes, and to design and optimize pharmacological approaches for selectively controlling these molecular drivers.

### SUMMARY

The present disclosure addresses the above-identified shortcomings. The present disclosure addresses these shortcomings, at least in part, using systems and methods of discovering a test compound that has a first biological property (e.g., an indication as to whether a compound activates or inhibits a cell state). A first training dataset is obtained, including chemical structures and biological properties. Projections of compounds are obtained by projecting chemical structure information into a latent representation space using encoder weights (e.g., a first plurality of weights associated with an untrained or partially untrained neural network encoder). Compounds are classified by inputting projections into the classifier using classifier weights (e.g., a second plurality of weights associated with an untrained or partially untrained classifier). The encoder and classifier are trained by comparing the classification of each compound to actual biological properties and updating the respective weights. A second training dataset is obtained including chemical structures. Projections of compounds are obtained by projecting chemical structure information into a latent representation space using encoder weights (e.g., the first plurality of weights associated with the trained neural network encoder). Chemical structures are obtained by inputting projections into a decoder using decoder weights (e.g., a third plurality of weights associated with an untrained or partially untrained decoder). The decoder is trained by comparing outputted and actual chemical structures and updating the respective weights. Candidate compounds (e.g., a test compound that has the first biological property) not present in the first and second datasets are identified using the trained encoder, classifier, and decoder.

One aspect of the present disclosure provides methods for discovering a test compound that has a first biological property. The method comprises, at a computer system comprising at least one processor and a memory storing at least one program for execution by the at least one processor, the at least one program comprising instructions for obtaining a first training dataset, in electronic form. The first training dataset comprises, for each respective compound in a first plurality of compounds (i) information regarding a chemical structure of the respective compound and (ii) one or more biological properties, in a plurality of biological properties, of the respective compound. The plurality of biological properties includes the first biological property.

An untrained or partially untrained neural network encoder and an untrained or partially untrained classifier is trained by performing a first procedure. For each respective compound in the first plurality of compounds, the information regarding the chemical structure of the respective compound is projected into a latent representation space according to a first plurality of weights associated with the untrained or partially untrained neural network encoder to obtain a corresponding projected representation of the respective compound. The corresponding projected representation of the respective compound is inputted into the untrained or partially untrained classifier to obtain a classification of the respective compound according to a second plurality of weights associated with the untrained or partially untrained classifier. The first plurality of weights and the second plurality of weights is updated by comparing the classification of each respective compound in the first plurality of compounds to the one or more biological properties of the respective compound in the first training dataset, thus obtaining a trained neural network encoder and a trained classifier.

A second training dataset is obtained, in electronic form, where the second training dataset comprises, for each respective compound in a second plurality of compounds, information regarding a chemical structure of the respective compound.

An untrained or partially untrained decoder is trained by performing a second procedure. For each respective compound in the second plurality of compounds, the information regarding the chemical structure of the respective compound is projected into a latent representation space according to the first plurality of weights associated with the trained neural network encoder to obtain a corresponding projected representation of the respective compound. The corresponding projected representation of the respective compound is inputted into the untrained or partially untrained decoder to obtain a chemical structure of the respective compound according to a third plurality of weights associated with the untrained or partially untrained decoder. The third plurality of weights is updated by comparing the chemical structure of each respective compound outputted by the untrained or partially untrained decoder to the actual chemical structure of the respective compound from the second training dataset, thus obtaining a trained decoder.

The trained neural network encoder, trained classifier, and trained decoder are used to identify a test compound that has the first biological property, where the test compound is not present in the first and second training set.

In some embodiments, the information regarding a chemical structure of the respective compound in the first plurality of compounds is a chemical structure of the respective compound or a high dimensional vector representation based upon a chemical structure of the respective compound.

In some embodiments, using the trained neural network encoder, trained classifier, and trained decoder comprises interpolating a projected representation of a first compound and a projected representation of a second compound, produced by the trained neural network encoder, where the first and second compound have the first molecular property thereby obtaining an interpolated projection. The interpolated projection is inputted into the trained decoder thereby obtaining a plurality of candidate compounds. For each respective candidate compound in all or a portion of the plurality of candidate compounds, a corresponding projected representation for the respective candidate compound is obtained by inputting a chemical structure of the candidate compound into the trained neural network encoder, and a classification of the respective candidate compound is obtained by inputting the corresponding projected representation of the respective candidate compound into the trained classifier. When the trained classifier indicates that the corresponding projected representation of the respective candidate compound has the first biological property, the respective candidate compound is deemed to have the first biological property.

In some such embodiments, the method further comprises verifying a first compound in the plurality of candidate compounds has the first biological property by a third procedure that comprises subjecting the first compound to a wet lab assay that verifies that the respective candidate compound has the first biological property. In some such embodiments, the method further comprises synthesizing the first compound.

In some embodiments, the method further comprises verifying the trained neural network encoder, trained classifier, and trained decoder by a third procedure that comprises obtaining a first compound, not present in the first or second training dataset, that has the first biological property and has a known chemical structure; obtaining a projected representation for the first compound by inputting a chemical structure of the first compound into the trained neural network encoder; inputting the projected representation of the first compound into the trained classifier to verify that the trained classifier identifies the first compound as having the first biological property; and inputting the projected representation of the first compound into the trained decoder to verify that the trained decoder reconstructs the chemical structure of the first compound.

In some embodiments, the information regarding the chemical structure of the respective compound is a molecular structure of the respective compound; the method further comprises forming a featurization of the chemical structure and incorporating the featurization of the chemical structure into a multi-dimensional vector space; and the projecting the information regarding the chemical structure of the respective compound into the latent representation space in accordance with the first plurality of weights associated with the untrained or partially untrained neural network encoder comprises inputting the multi-dimensional vector space of the chemical structure into the untrained or partially untrained neural network encoder.

In some embodiments, the featurization of the chemical structure is a tensor. In some such embodiments, the tensor is a one-dimensional vector or a two-dimensional matrix.

In some embodiments, the featurization of the chemical structure is an extended circular fingerprint, or a molecular graph of a plurality of one-hot-encoded vectors.

In some embodiments, the multi-dimensional vector space is an N-dimensional space, where N is an integer between 20 and 80. In some embodiments, N is 50.

In some embodiments, the incorporating the featurization of the chemical structure into the multi-dimensional vector space for the chemical structure comprises inputting the featurization of the chemical structure into a spatial graph convolutional network (GCN). In some embodiments, the GCN is a graph attention network (GAT), a graph isomorphism network (GIN), or a graph substructure index-based approximate graph (SAGA).

In some embodiments, the incorporating the featurization of the molecular structure into the multi-dimensional vector space for the chemical structure comprises an application of a spectral graph convolution (SGC) to the featurization of the chemical structure. In some embodiments, the application of the SGC to the featurization of the chemical structure uses Chebyshev polynomial filtering.

In some embodiments, the forming the featurization of the chemical structure comprises converting the chemical structure to a simplified molecular-input line-entry system (SMILES) string, and converting the SMILES string into a molecular graph representation that comprises an adjacency matrix and a feature matrix.

In some embodiments, the first biological property is selected from the group consisting of: an indication as to whether a compound activates a cell state, an indication as to whether a compound inhibits a cell state, an affinity for a biological target, an EC50 of the compound for inhibiting a biological state, an IC50 of the compound for inhibiting a biological state, an ED50 of the compound for inhibiting a biological state, an LD50 of the compound for inhibiting a biological state, and a TD50 of the compound for inhibiting a biological state.

In some embodiments, the cell state is characterized by an up-regulation or down-regulation of one or more respective genes in a plurality of genes associated with the cell state. In some embodiments, the cell state is a diseased state. In some embodiments, the cell state is characterized by an upregulation or a down-regulation of one or more biological pathways. In some embodiments, the cell state is characterized by an upregulation or a down-regulation of one or more biological pathways in a plurality of biological pathways.

In some embodiments, the cell state is characterized by an upregulation or a down-regulation of one or more cellular-components. In some such embodiments, the one or more cellular-components comprises a plurality of genes, optionally measured at the RNA level. In some embodiments, the one or more cellular-components are quantified using single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, or any combinations thereof, or summaries of the same, including combinations, such as linear combinations, representing activated pathways in the single-cell cellular-component expression datasets. In some embodiments, the one or more cellular-components comprises a plurality of proteins.

Another aspect of the present disclosure provides a method of discovering a candidate compound that has a first biological property. The method comprises, at a computer system comprising at least one processor and a memory storing at least one program for execution by the at least one processor, the at least one program comprising instructions for obtaining a first projected representation of a first compound that is assigned the first biological property by inputting a chemical structure of the first compound into a trained neural network encoder (e.g., where the first projected representation has N dimensions and N is an integer between 20 and 80).

The first projection is used to obtain one or more candidate projections. Each candidate projection in the one or more candidate projections is inputted into a trained decoder thus obtaining a plurality of candidate compounds, where the first compound is not present in the plurality of candidate compounds. For each respective candidate compound in the plurality of candidate compounds, a corresponding projected representation (e.g., an N-dimensional projected representation) for the respective candidate compound is obtained by inputting a chemical structure of the candidate compound into the trained neural network encoder, and a classification of the respective candidate compound is obtained by inputting the corresponding projected representation of the respective candidate compound into the trained classifier. When the trained classifier indicates that the corresponding projected representation of the respective candidate compound has the first biological property, the respective candidate compound is deemed to have the first biological property.

In some embodiments, the method further comprises obtaining a second projected representation of a second compound that has the biological property by inputting a chemical structure of the second compound into the trained neural network encoder, and the using the first projection to obtain one or more candidate projections comprises interpolating the first projection and the second projection thus obtaining the one or more candidate projections.

In some embodiments, the first biological property is a compound function.

In some embodiments, the method further comprises subjecting the respective candidate compound to a wet lab assay that verifies that the respective candidate compound has the first biological property. In some embodiments, the method further comprises synthesizing the respective candidate compound.

Another aspect of the present disclosure provides a method of discovering a test compound that has a first biological property. The method comprises at a computer system comprising at least one processor and a memory storing at least one program for execution by the at least one processor, the at least one program comprising instructions for using a trained neural network encoder, trained classifier, and trained decoder to identify a test compound that has a first biological property, where the test compound is not present in a first and second training set.

In this aspect, the trained neural network encoder, trained classifier, and trained decoder were trained by processes comprising obtaining a first training dataset, in electronic form, where the first training dataset comprises, for each respective compound in a first plurality of compounds (e.g., comprising 100 or more compounds), information regarding a chemical structure of the respective compound and one or more biological properties, in a plurality of biological properties, of the respective compound. The plurality of biological properties includes the first biological property.

The processes further comprise training an untrained or partially untrained neural network encoder and an untrained or partially untrained classifier by performing a first procedure that comprises, for each respective compound in the first plurality of compounds, projecting the information regarding the chemical structure of the respective compound into a latent representation space according to a first plurality of weights associated with the untrained or partially untrained neural network encoder to obtain a corresponding projected representation of the respective compound, and inputting the corresponding projected representation of the respective compound into the untrained or partially untrained classifier to obtain a classification of the respective compound according to a second plurality of weights associated with the untrained or partially untrained classifier. The first procedure further comprises updating the first plurality of weights and the second plurality of weights by comparing the classification of each respective compound in the first plurality of compounds to the one or more biological properties of the respective compound in the first training dataset thus obtaining a trained neural network encoder and a trained classifier.

The processes further comprise obtaining a second training dataset, in electronic form, where the second training dataset comprises, for each respective compound in a second plurality of compounds (e.g., comprising 100 or more compounds), information regarding a chemical structure of the respective compound.

The processes further comprise training an untrained or partially untrained decoder by performing a second procedure that comprises for each respective compound in the second plurality of compounds, projecting the information regarding the chemical structure of the respective compound into a latent representation space according to the first plurality of weights associated with the trained neural network encoder to obtain a corresponding projected representation of the respective compound, and inputting the corresponding projected representation of the respective compound into the untrained or partially untrained decoder to obtain a chemical structure of the respective compound according to a third plurality of weights associated with the untrained or partially untrained decoder. The second procedure further comprises updating the third plurality of weights by comparing the chemical structure of each respective compound outputted by the untrained or partially untrained decoder to the actual chemical structure of the respective compound from the second training dataset thus obtaining a trained decoder.

Another aspect of the present disclosure provides a method of synthesizing a test compound that has a first biological property, where the test compound was designed by a method. The method comprises, at a computer system comprising at least one processor and a memory storing at least one program for execution by the at least one processor, at least one program comprising instructions for obtaining a first training dataset, in electronic form. The first training dataset comprises, for each respective compound in a first plurality of compounds (e.g., comprising 100 or more compounds), information regarding a chemical structure of the respective compound and one or more biological properties, in a plurality of biological properties, of the respective compound, and the plurality of biological properties includes the first biological property.

In this aspect, the method further comprises training an untrained or partially untrained neural network encoder and an untrained or partially untrained classifier by performing a first procedure. The first procedure comprises, for each respective compound in the first plurality of compounds, projecting the information regarding the chemical structure of the respective compound into a latent representation space in accordance with a first plurality of weights associated with the untrained or partially untrained neural network encoder to obtain a corresponding projected representation of the respective compound, and inputting the corresponding projected representation of the respective compound into the untrained or partially untrained classifier to obtain a classification of the respective compound in accordance with a second plurality of weights associated with the untrained or partially untrained classifier. The first procedure further comprises updating the first plurality of weights and the second plurality of weights by comparing the classification of each respective compound in the first plurality of compounds to the one or more biological properties of the respective compound in the first training dataset thereby obtaining a trained neural network encoder and a trained classifier.

The method further comprises obtaining a second training dataset, in electronic form, where the second training dataset comprises, for each respective compound in a second plurality of compounds (e.g., comprising 100 or more compounds), information regarding a chemical structure of the respective compound. The method further comprises training an untrained or partially untrained decoder by performing a second procedure that comprises, for each respective compound in the second plurality of compounds, projecting the information regarding the chemical structure of the respective compound into a latent representation space in accordance with the first plurality of weights associated with the trained neural network encoder to obtain a corresponding projected representation of the respective compound, and inputting the corresponding projected representation of the respective compound into the untrained or partially untrained decoder to obtain a chemical structure of the respective compound in accordance with a third plurality of weights associated with the untrained or partially untrained decoder. The second procedure further comprises updating the third plurality of weights by comparing the chemical structure of each respective compound outputted by the untrained or partially untrained decoder to the actual chemical structure of the respective compound from the second training dataset thereby obtaining a trained decoder.

The method further comprises using the trained neural network encoder, trained classifier, and trained decoder to identify a test compound that has the first biological property, wherein the test compound is not present in the first and second training set.

In some embodiments, the method of synthesizing a test compound that has a first biological property further comprises any of the methods for discovering a test compound that has a first biological property described in the present disclosure.

Another aspect of the present disclosure provides a computer system, comprising one or more processors and memory, the memory storing instructions for performing any of the methods for discovering a test compound that has a first biological property described in the present disclosure.

Yet another aspect of the present disclosure provides a non-transitory computer-readable medium storing one or more computer programs, executable by a computer, for performing a method for discovering a test compound that has a first biological property, the computer comprising one or more processors and a memory, the one or more computer programs collectively encoding computer executable instructions, which when executed by a computer system, cause the computer system to perform any of the methods for discovering a test compound that has a first biological property described in the present disclosure.

### BRIEF DESCRIPTION OF THE DRAWINGS

The embodiments disclosed herein are illustrated by way of example, and not by way of limitation, in the figures of the accompanying drawings. Like reference numerals refer to corresponding parts throughout the drawings.

FIG. 1 illustrates a block diagram of an exemplary system and computing device for discovering a compound that has a biological property, in accordance with an embodiment of the present disclosure;

FIGS. 2A and 2B provide a flow chart of processes for discovering a compound that has a biological property, in accordance with various embodiments of the present disclosure, in which elements in dashed boxes are optional:

FIG. 3 illustrates molecular design, in accordance with an embodiment of the present disclosure;

FIG. 4 illustrates molecular design and optimization, in accordance with an embodiment of the present disclosure:

FIG. 5 illustrates steps of compound generation, in accordance with an embodiment of the present disclosure;

FIG. 6 illustrates creating a label for a compound from score distribution, in accordance with an embodiment of the present disclosure:

FIG. 7 illustrates loss curves during the training of a neural network encoder and a classifier, where the neural network encoder and the classifier are converged without overfitting, in accordance with an embodiment of the present disclosure:

FIG. 8 illustrates the precision at 10% recall scores during the training of a neural network encoder and a classifier for example pathways, in accordance with an embodiment of the present disclosure;

FIG. 9 illustrates an example of encoded molecular representation space, in accordance with an embodiment of the present disclosure; and

FIGS. 10A-D collectively illustrate molecules that promote arachidonic acid metabolism. In FIG. 10A, molecules that promote arachidonic acid metabolism are sorted by their scores from a classifier of the present disclosure, in which generated molecules that are not found in the database used to train the classifier are shown in boxes, in accordance with an embodiment of the present disclosure. In FIGS. 10B, 10C, and 10D, generated molecules that are not found in the database used to train the classifier are shown in greater detail.

FIGS. 11A-L collectively illustrate the performance of a classification model for predicting compounds for each of 12 functional pathways, where the performance is measured as the precision at 10% recall score. 11A: Activating arachidonic acid metabolism; 11B: Inhibiting alpha-linolenic acid metabolism; 11C: Activating insulin secretion; 11D: Activating proteasome; 11E: Activating synaptic vesicle cycle; 11F: Inhibiting human T-cell leukemia virus 1 infection; 11G: Activating cytosolic DNA sensing pathway; 11H: Inhibiting calcium signaling pathway; 11I: Inhibiting Chagas disease (e.g., American trypanosomiasis); 11J: Inhibiting oocyte meisosis; 11K: Inhibiting nucleotide excision repair; 11L: Activating pancreatic secretion.

### DETAILED DESCRIPTION

Tissues are complex ecosystems of individual cells, where dysregulation of cell state is the basis of disease. Existing drug discovery efforts seek to characterize the molecular mechanisms that cause cells to transition from healthy to disease states, and to identify pharmacological approaches to reverse or inhibit these transitions. Past efforts have also sought to identify molecular signatures characterizing these transitions, and to identify pharmacological approaches that reverse these signatures.

Several difficulties, however, emerge in this pursuit. For example, there exist a vast number of “drug-like” molecules (e.g., on the order of 10⁶⁰ molecules) that can serve as potential drug candidates. Among these, identifying the few target chemical compounds that effect biological change (e.g., disrupting functional pathways, inhibiting transitions from healthy to disease states and/or resolving disease mechanisms) is difficult and laborious, traditionally requiring extensive experimentation and/or prior knowledge of chemical compounds and their biological properties. In particular, current approaches for engineering molecules for drug discovery are expensive, slow, and inefficient, for example where a drug discovery assay focuses on a single biological target or disease due to the complexity and number of targets for validation. Additionally, any molecules that are identified as potential drug candidates that interact with the desired biological target must be further optimized to remove or reduce any unwanted interactions leading to harmful side effects.

An alternative to experimental lead identification is to use computational, data-driven approaches. Among these, deep generative models are attractive approaches due to the ability to “learn” properties of molecular structure during training and subsequently perform automated generation of new synthetic structures with similar properties and any desired combinations thereof. However, conventional methods using generative models for chemical design largely focus on physical properties without considering the holistic effects of a generated molecule on the function and activity of one or more target biological processes, target cell states, or target cell state transitions. Additionally, these approaches frequently require prior knowledge of compound-target interactions, biological activity data for the candidate compounds, and/or annotations (e.g., characterizing molecular signatures and/or gene expression data specific to diseased cell state transitions). See, e.g., Lucio el al., 2020, “De novo generation of hit-like molecules from gene expression signatures using artificial intelligence,” Nature Comm. 11:10, doi:10.1038/s41467-019-13807-w.

The instant application addresses the shortcomings in the art, at least in part, by providing, inter alia, systems and methods for discovering molecules (sometimes referred to herein as a test compound) that have at least a first biological property (e.g., an indication as to whether a compound activates or inhibits a cell state).

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Plural instances may be provided for components, operations or structures described herein as a single instance. Finally, boundaries between various components, operations, and data stores are somewhat arbitrary, and particular operations are illustrated in the context of specific illustrative configurations. Other forms of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first dataset could be termed a second dataset, and, similarly, a second dataset could be termed a first dataset, without departing from the scope of the present invention. The first dataset and the second dataset are both datasets, but they are not the same dataset.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

As used interchangeably herein, a cell state or biological state refers to a state or phenotype of a cell or a population of cells. For example, a cell state may be healthy or diseased. A cell state may be characterized by a measure of one or more cellular-components, including but not limited to one or more genes, one or more proteins, and/or one or more biological pathways.

As used herein, a cell state transition or cellular transition refers to a transition in a cell's state from a first cell state to an altered cell state (e.g., healthy to diseased). A cell state transition can be marked by a change in cellular-component expression in the cell, and thus by the identity and quantity cellular-components (e.g., mRNA, transcription factors) produced by the cell.

As used herein, a perturbation refers to a treatment (e.g., of a cell) with one or more compounds. The one or more compounds can include, for example, a small molecule, a biologic, a protein, a protein combined with a small molecule, an ADC (antibody drug conjugate), a nucleic acid, such as an siRNA or interfering RNA, an aptamer, a cDNA over-expressing wild-type and/or mutant shRNA, a cDNA over-expressing wild-type and/or mutant guide RNA (e.g., Cas9 system or other cellular-component editing system), or any combination of any of the foregoing.

As used interchangeably herein, a latent representation space, high-dimensional representation space, multi-dimensional representation space, or latent vector space refers to a mathematical space where high-dimensional representations of compounds are projected. The high-dimensional representation may be a representation of a chemical structure, such as a SMILES string, which is projected into a vector representation by a neural network encoder.

### I. EXEMPLARY SYSTEM EMBODIMENTS

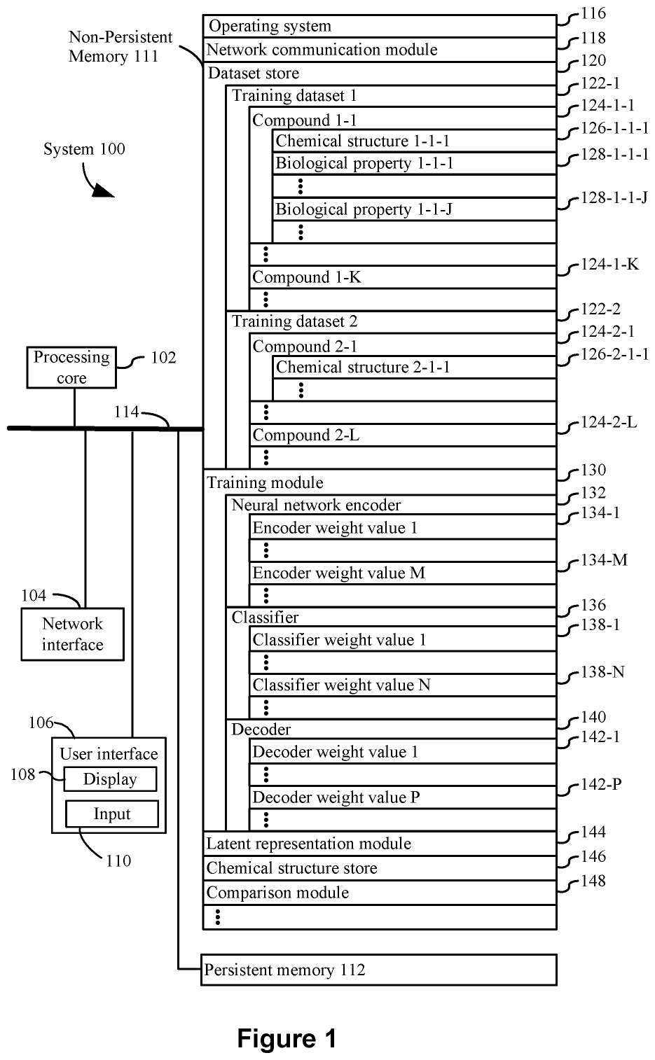

Now that an overview of some aspects of the present disclosure and some definitions used in the present disclosure have been provided, details of an exemplary system are described in conjunction with FIG. 1.

FIG. 1 provides a block diagram illustrating a system 100 in accordance with some embodiments of the present disclosure. The system 100 provides discovering a test compound that has a first biological property. In FIG. 1, the system 100 is illustrated as a computing device. In some embodiments, other topologies of the computer system 100 are possible. For instance, in some embodiments, the system 100 can in fact constitute several computer systems that are linked together in a network, or be a virtual machine or a container in a cloud computing environment. As such, the exemplary topology shown in FIG. 1 merely serves to describe the features of an embodiment of the present disclosure in a manner that will be readily understood to one of skill in the art.

Referring to FIG. 1, in some embodiments a computer system 100 (e.g., a computing device) includes a network interface 104. In some embodiments, the network interface 104 interconnects the system 100 computing devices within the system with each other, as well as optional external systems and devices, through one or more communication networks (e.g., through network communication module 118). In some embodiments, the network interface 104 optionally provides communication through network communication module 118 via the Internet, one or more local area networks (LANs), one or more wide area networks (WANs), other types of networks, or a combination of such networks.

Examples of networks include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP). Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

The system 100 in some embodiments includes one or more processing units (CPU(s)) 102 (e.g., a processor, a processing core, etc.), one or more network interfaces 104, a user interface 106 including (optionally) a display 108 and an input system 110 (e.g. an input/output interface, a keyboard, a mouse, etc.) for use by the user, memory (e.g., non-persistent memory 111, persistent memory 112), and one or more communication buses 114 for interconnecting the aforementioned components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, include non-transitory computer readable storage medium. In some embodiments, the non-persistent memory 111 or alternatively the non-transitory computer readable storage medium stores the following programs, modules and data structures, or a subset thereof, sometimes in conjunction with the persistent memory 112:
an optional operating system 116 (e.g., ANDROID, iOS, DARWIN, RTXC, LINUX, UNIX, OS X, WINDOWS, or an embedded operating system such as VxWorks), which includes procedures for handling various basic system services and for performing hardware dependent tasks;
an optional network communication module (or instructions) 118 for connecting the system 100 with other devices and/or a communication network 104;
a dataset store 120 that stores a first training dataset 122-1 including a first plurality of compounds 124 (e.g., 124-1-1, . . . , 124-1-K) comprising, for each respective compound in the first plurality of compounds in the first dataset, information regarding a chemical structure of the respective compound 126 (e.g., 126-1-1-1) and one or more biological properties 128 (e.g., 128-1-1-1, . . . , 128-1-1-J) in a plurality of biological properties, and a second training dataset 122-2 including a second plurality of compounds 124 (e.g., 124-2-1, . . . , 124-2-L) comprising, for each respective compound in the second plurality of compounds in the second dataset, information regarding a chemical structure of the respective compound 126 (e.g., 126-2-1-1);
a training module 130 comprising a neural network encoder 132 including a first plurality of weights associated with the neural network encoder 134 (e.g., 134-1, . . . 134-M), a classifier 136 including a second plurality of weights associated with the classifier 138 (e.g., 138-1, . . . , 138-N), and a decoder 140 including a third plurality of weights associated with the decoder 142 (e.g., 142-1, . . . , 142-P);
a latent representation module 144 that, upon projecting the information regarding the chemical structure of a respective compound in accordance with a first plurality of weights associated with the neural network encoder, generates a corresponding projected representation of the respective compound;
a chemical structure store 146 that stores the chemical structure of a respective compound outputted by the decoder; and
a comparison module 148 that compares the classification of each respective compound in the first plurality of compounds to the one or more biological properties of the respective compound in the first training dataset to update the first plurality of weights 134 and the second plurality of weights 138, and compares the chemical structure of each respective compound outputted by the decoder to the actual chemical structure of the respective compound from the second training dataset to update the third plurality of weights 142.

As described above, the dataset store 120 includes a first training dataset 122-1 and a second training dataset 122-2. Each dataset is obtained (e.g., collected, communicated, etc.) in electronic form. The training module comprises the neural network encoder 132, the classifier 136, and the decoder 140, each of which comprise a respective plurality of weights that are used to obtain a result from an input. For example, the neural network encoder projects the information regarding the chemical structure of a respective compound 126 into a latent representation space in accordance with the first plurality of weights associated with the neural network encoder to obtain a corresponding projected representation of the respective compound (e.g., via the latent representation module 144). Additionally, the classifier uses the corresponding projected representation of the respective compound to obtain a classification of the respective compound in accordance with the second plurality of weights associated with the classifier. Furthermore, the decoder uses a corresponding projected representation of a respective compound to obtain a chemical structure of the respective compound in accordance with the third plurality of weights associated with the decoder. Chemical structures obtained using the decoder can be stored in the chemical structure store 146, for example, for further comparison via the comparison module 148.

The respective plurality of weights in the neural network encoder 132, the classifier 136, and/or the decoder 140 are updated as a result of comparison results obtained from the comparison module 148 (e.g., via back-propagation). As a result, in some embodiments, the neural network encoder, the classifier and the decoder is untrained, partially untrained, or trained based on the values of the respective plurality of weights. A trained neural network encoder, trained classifier, and trained decoder are subsequently used to identify a test compound that has the first biological property, where the identified test compound is not previously present in the first and second training datasets.

In various embodiments, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of the system 100, that is addressable by the system 100 so that the system 100 may retrieve all or a portion of such data when needed.

Although FIG. 1 depicts a “system 100.” the figure is intended more as a functional description of the various features that may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules instead may be stored in persistent memory 112 or in more than one memory. For example, in some embodiments, at least dataset store 120 is stored in a remote storage device which can be a part of a cloud-based infrastructure. In some embodiments, at least dataset store 120 is stored on a cloud-based infrastructure. In some embodiments, dataset store 120 and chemical structure store 146 can both be stored in the remote storage device(s) and/or the cloud-based infrastructure.

### II. SPECIFIC EMBODIMENTS OF THE DISCLOSURE

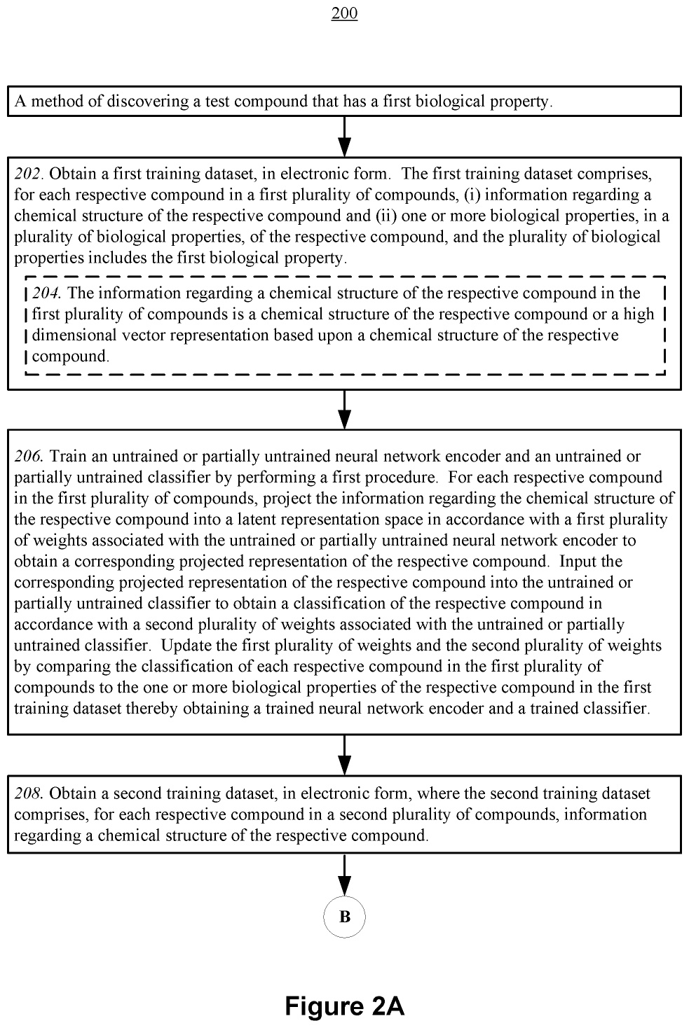

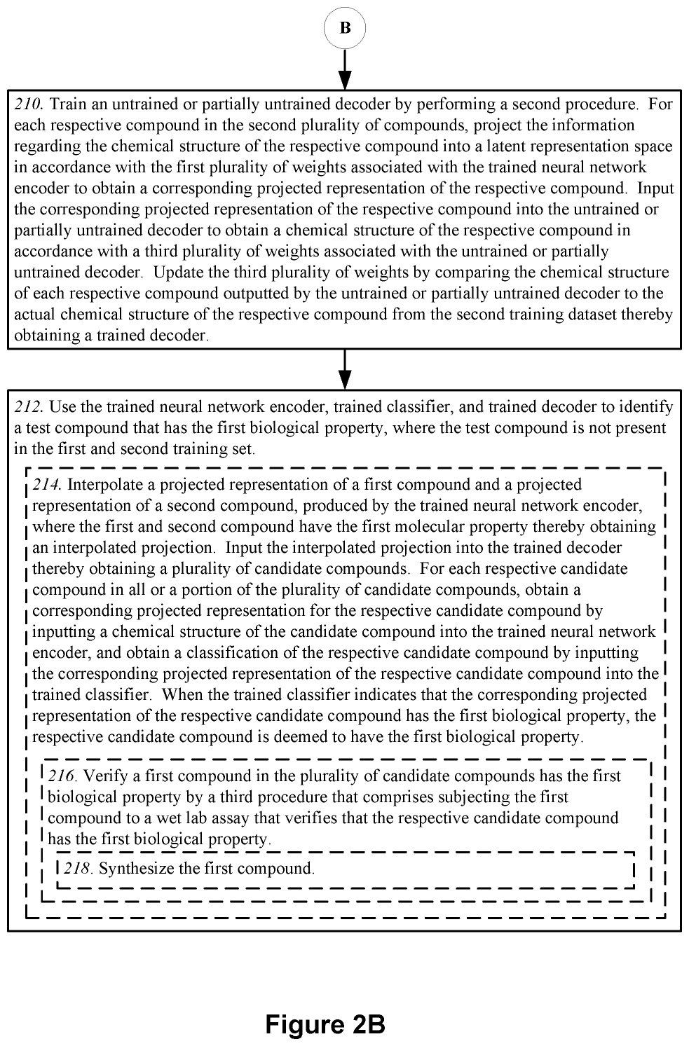

While a system in accordance with the present disclosure has been disclosed with reference to FIG. 1, a method of discovering a test compound that has a first biological property 200 in accordance with one aspect of the present disclosure is now detailed with reference to FIG. 2.

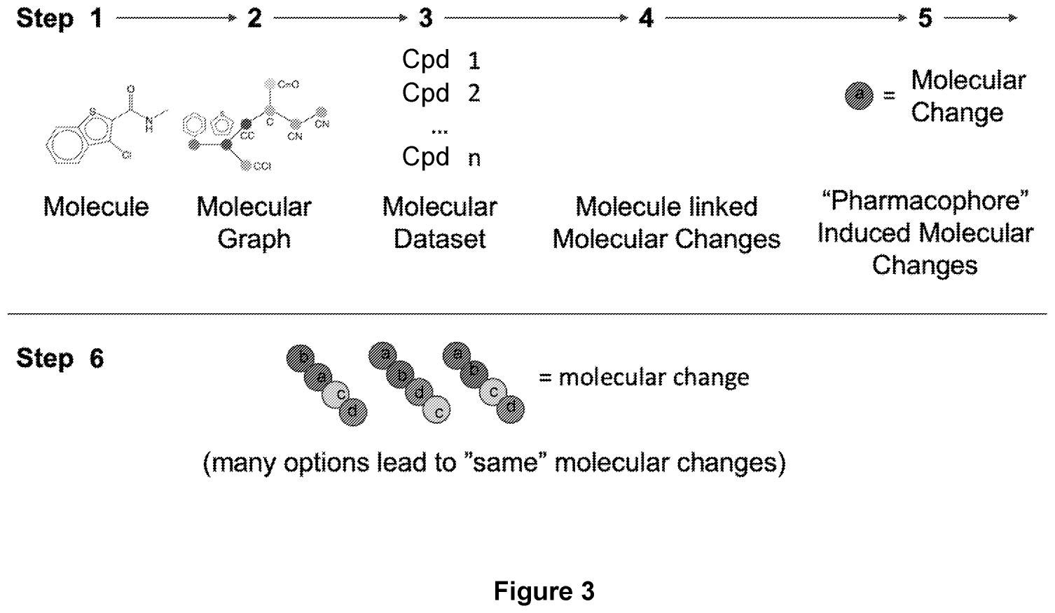

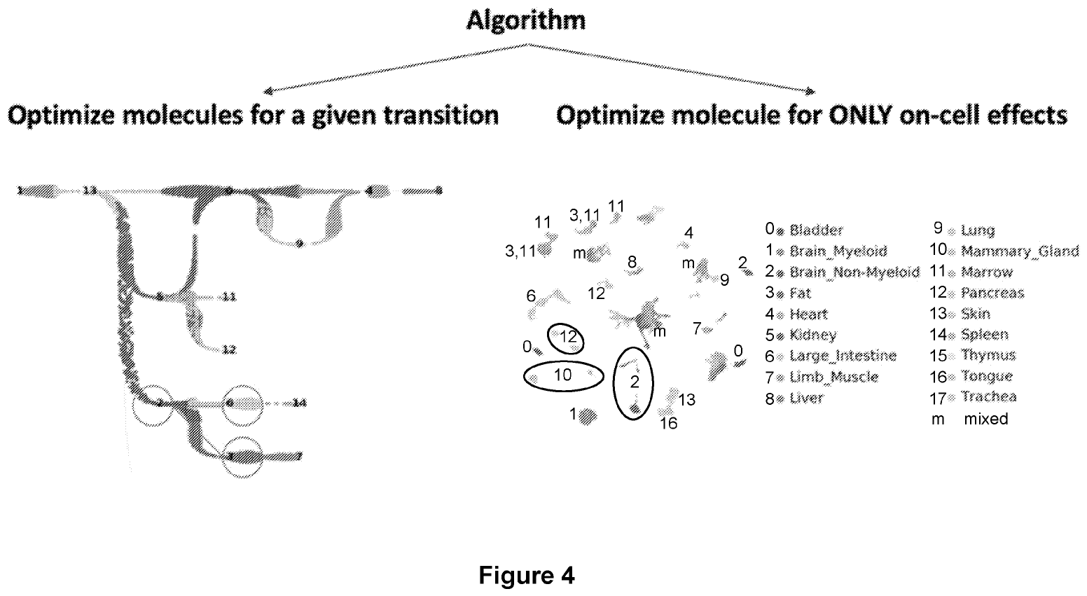

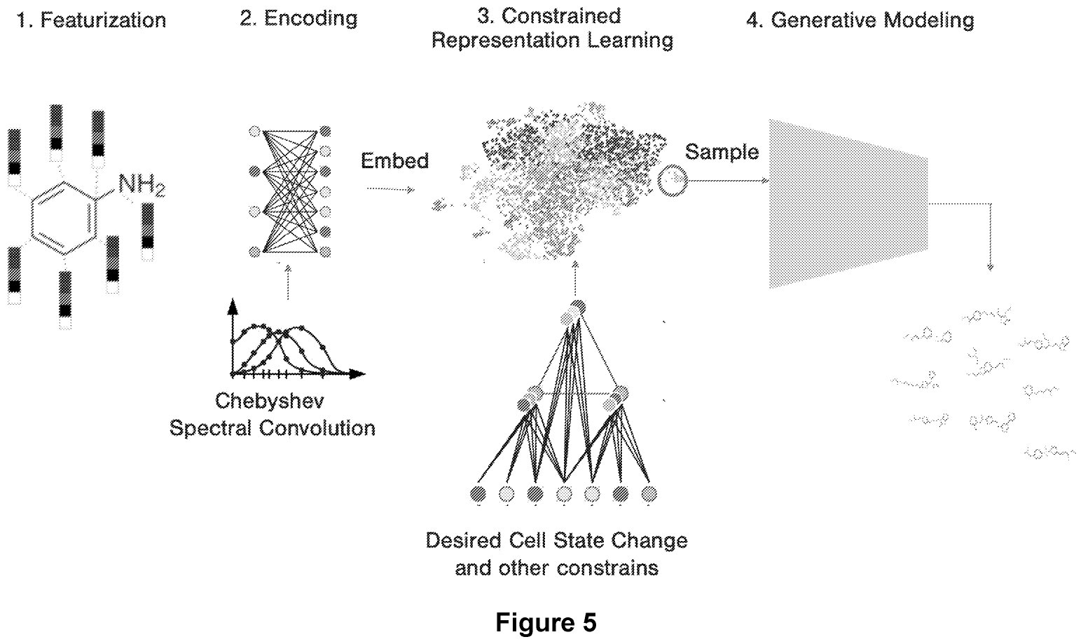

As illustrated in FIGS. 3, 4, and 5, by computationally optimizing molecules for their biological properties (e.g., their effects on cell states), molecules that have a high probability of having a desired biological property (e.g., producing a desired biological effect) without having undesired effects at cellular level are generated. An important benefit is the ability of this approach to concurrently generate molecules optimized for several targets, biological states and diseases. In some embodiments, the machine learning-driven molecular optimization involves two phases, e.g., training and inference, and four steps: featurization, embedding (e.g., molecule structure encoding), constrained representation learning, and generation (e.g., molecule generation).

Datasets

Referring to Block 202, the method comprises, at a computer system comprising at least one processor and a memory storing at least one program for execution by the at least one processor, the at least one program comprising instructions for obtaining a first training dataset, in electronic form. The first training dataset comprises, for each respective compound in a first plurality of compounds (e.g., comprising 100 or more compounds), information regarding a chemical structure of the respective compound and one or more biological properties, in a plurality of biological properties, of the respective compound. The plurality of biological properties includes the first biological property.

In some embodiments, the first training dataset comprises virtual compounds. In some embodiments, the first training dataset is a small molecules and/or ligand dataset. In some embodiments, the first training dataset is all or a portion of a Library of Integrated Network-based Cellular Signatures (LINCS) L1000 dataset. In some embodiments, the first plurality of compounds comprises at least 10, at least 20, at least 25, at least 30, at least 35, at least 40, at least 45, at least 50, at least 55, at least 60, at least 65, at least 70, at least 75, at least 80, at least 85, at least 90, at least 95, or at least 100 compounds. In some embodiments, the first plurality of compounds comprises at least 50, at least 100, at least 200, at least 300, at least 400, at least 500, at least 600, at least 700, at least 800, at least 900, or at least 1000 compounds. In some embodiments, the first plurality of compounds comprises at least 1000, at least 2000, at least 3000, at least 4000, at least 5000, at least 10,000, at least 100,000, or at least 1 million compounds. In some embodiments, the first plurality of compounds comprises no more than 10, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 compounds. In some embodiments, the first plurality of compounds comprises no more than 50, no more than 100, no more than 200, no more than 300, no more than 400, no more than 500, no more than 600, no more than 700, no more than 800, no more than 900, or no more than 1000 compounds. In some embodiments, the first plurality of compounds comprises no more than 1000, no more than 2000, no more than 3000, no more than 4000, no more than 5000, no more than 10,000, no more than 100,000, no more than 1 million, no more than 2 million, no more than 5 million, or no more than 10 million compounds. In some embodiments, the first plurality of compounds comprises between 2 and 20, between 20 and 50, between 50 and 100, between 100 and 200, between 200 and 500, between 500 and 1000, between 1000 and 5000, between 5000 and 10,000, between 10,000 and 100,000, between 100,000 and 1 million, or between 1 and 5 million compounds. In some embodiments, the first training dataset comprises 100 or more, 1,000 or more, 10,000 or more, 100,000 or more, 250,000 or more, 500,000 or more, 1 million or more, 2 million or more, or 5 million or more compounds. In some embodiments, the first training dataset comprises information regarding one or more biological and/or functional pathways for each respective compound in the first plurality of compounds.

Referring to Block 204, in some embodiments, the information regarding a chemical structure of the respective compound in the first plurality of compounds is a chemical structure of the respective compound or a high dimensional vector representation based upon a chemical structure of the respective compound.

In some embodiments, the information regarding a chemical structure of the respective compound in the first plurality of compounds is a simplified molecule-input line-entry system (SMILES). A SMILES string is a method of encoding and/or representing molecular structures as a 1-dimensional vector or string. See, e.g., EPA, 2012, “SMILES Notation Tutorial,” Sustainable Futures/P2 Framework Manual EPA-748-B12-001, Appendix F.

In some embodiments, the first biological property is a compound function, that is, a combination, such as a linear combination, of two or more functions.

In some embodiments, the first biological property is selected from the group consisting of: an indication as to whether a compound activates a cell state, an indication as to whether a compound inhibits a cell state, an affinity for a biological target, an EC50 of the compound for inhibiting a biological state, an IC50 of the compound for inhibiting a biological state, an ED50 of the compound for inhibiting a biological state, an LD50 of the compound for inhibiting a biological state, a TD50 of the compound for inhibiting a biological state, and/or a concentration of the compound at 50% activity for a biological state (e.g., inhibiting a particular biological pathway).

In some embodiments a biological property is a measure of toxicity. For example, in some embodiments a biological property is inhibition or activation of a nuclear receptor. As another example, in some embodiments a biological property is an amount of inhibition or an amount of activation of a nuclear receptor. In some embodiments a biological property is an amount of inhibition or an amount of activation of a stress response pathway. Example nuclear receptors and example stress response pathways, as well as inhibition or activation data for these nuclear receptors and example stress response pathways that can be used in the present disclosure, are described for approximately 10,000 compounds as described in Huang et al. 2016, “Modelling the Tox21 10 K chemical profiles for in vivo toxicity prediction and mechanism characterization,” Nat Commun. 7, p. 10425, which is hereby incorporated by reference.

In some embodiments, a biological property is a measure of solubility (e.g., c Log P). In some embodiments, a biological property is a measure of pharmacological activity or druglikeness (e.g., Lipinski's rule of five). For example, in some embodiments, a biological property is a measure of one or more of absorption, distribution, metabolism, and/or excretion in a biological organism (e.g., a human body). In some embodiments, biological properties are measured by any assay known in the art, including, but not limited to, colorimetric, fluorescence, luminescence (e.g., bioluminescence), and resonance energy transfer (FRET). In some embodiments, biological properties are measured using high-throughput screening (HTS) and/or high-content screening (HCS) methods. Other methods for measuring and/or biological properties are contemplated, for example as described in Huang R, 2016, “A Quantitative High-Throughput Screening Data Analysis Pipeline for Activity Profiling,” High-Throughput Screening Assays in Toxicology, Methods in Molecular Biology; 1473(1); Huang et al., 2016, “Modelling the Tox21 10 K chemical profiles for in vivo toxicity prediction and mechanism characterization.” Nat Commun. 7, p. 10425, and Huang et al., 2018, “Expanding biological space coverage enhances the prediction of drug adverse effects in human using in vitro activity profiles,” Sci Rep. 8(1):3783, each of which is hereby incorporated herein by reference in its entirety, and/or any substitutions, additions, deletions, modifications, and/or combinations thereof as will be apparent to one skilled in the art.

In some embodiments, the plurality of biological properties comprises 1, 2, 3, 4, 5, 6, 7, 8, 9, 10, 11, 12, 13, 14, 15, 16, 17, 18, 19, 20, or more than 20 biological properties. In some embodiments, the plurality of biological properties comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 biological properties. In some embodiments, the plurality of biological properties comprises between 1 and 5, between 5 and 10, between 10 and 20, between 20 and 30, between 30 and 40, between 40 and 50, or between 50 and 100 biological properties.

In some embodiments, the cell state (e.g., a cell state activated and/or inhibited by a respective compound) is characterized by an up-regulation or down-regulation of one or more respective genes in a plurality of genes associated with the cell state. In some embodiments, the cell state is a diseased state.

In some embodiments, the cell state is characterized by an upregulation or a down-regulation of one or more biological pathways. In some embodiments, the cell state is characterized by an upregulation or a down-regulation of one or more biological pathways in a plurality of biological pathways. In some embodiments a biological pathway in the plurality of biological pathways is represented in the KEGG pathway database available on the Internet at www.genome.jp/kegg/pathway.html.

In some embodiments, the cell state is characterized by an upregulation or a down-regulation of one or more cellular-components.

For example, in some embodiments, cell state transitions (i.e., a transition in a cell's state from a first cell state to an altered cell state) are marked by a change in expression of cellular-components in the cell. For example, a transition can be marked by a change in cellular-component expression in the cell, and thus by the identity and quantity cellular-components (e.g., mRNA, transcription factors) produced by the cell.

As another example, in some embodiments, the one or more cellular-components comprises a plurality of genes, optionally measured at the RNA level. In some embodiments, the plurality of genes comprises at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 genes. In some embodiments, the plurality of genes comprises at least 50, at least 100, at least 200, at least 300, at least 400, at least 500, at least 600, at least 700, at least 800, at least 900, or at least 1000 genes. In some embodiments, the plurality of genes comprises at least 1000, at least 2000, at least 3000, at least 4000, at least 5000, at least 10,000, at least 30,000, at least 50,000, or more than 50,000 genes. In some embodiments, the plurality of genes comprises between 2 and 20, between 20 and 50, between 50 and 100, between 100 and 200, between 200 and 500, between 500 and 1000, between 1000 and 5000, between 5000 and 10,000 genes, or between 10,000 and 50.000 genes. In some embodiments, the one or more cellular-components comprises a plurality of proteins. In some embodiments, the plurality of proteins comprises at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 proteins. In some embodiments, the plurality of proteins comprises at least 50, at least 100, at least 200, at least 300, at least 400, at least 500, at least 600, at least 700, at least 800, at least 900, or at least 1000 proteins. In some embodiments, the plurality of proteins comprises at least 1000, at least 2000, at least 3000, at least 4000, at least 5000, at least 10,000, at least 30,000, at least 50,000, or more than 50,000 proteins. In some embodiments, the plurality of proteins comprises between 2 and 20, between 20 and 50, between 50 and 100, between 100 and 200, between 200 and 500, between 500 and 1000, between 1000 and 5000, between 5000 and 10,000 proteins, or between 10,000 and 50,000 proteins. In some embodiments, cellular-components of interest include nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc. proteins), lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular-components such as oxidized and reduced forms of nicotinamide adenine dinucleotide (NADP/NADPH), and any combinations thereof.

For example, in some embodiments, a cellular-component is selected from the group consisting of AhR, AP-1, AR-BLA, ARE, AR-MDA, aromatase, CAR, caspases (e.g., caspase-3/7), ATAD5, ER-beta, ER-BLA, ER-BG1, ERR, ER stress, FXR-BLA, TR-beta, GR-BLA, H2AX, HDAC, HRE-BLA, HSE-BLA, NFkB, P53, PGC-ERR, PPAR-delta-BLA, PPAR-gamma, PR-BLA, PXR, RAR, ROR, RXR-BLA, SBE-BLA (TGF-beta), Hedgehog, TRHR, TSHR, VDR-BLA, and/or any agonists and/or antagonists thereof as will be apparent to one skilled in the art.

In some embodiments, a cell state is determined based upon a change in cytotoxicity, cell viability, gene toxicity, developmental toxicity, and/or mitochondrial toxicity in response to an agonism and/or antagonism of one or more cellular-components of interest. Further examples of cellular-components, cell states, and/or methods for measuring the same are described in Huang R, 2016, “A Quantitative High-Throughput Screening Data Analysis Pipeline for Activity Profiling,” High-Throughput Screening Assays in Toxicology, Methods in Molecular Biology; 1473(1); Huang et al., 2016, “Modelling the Tox21 10 K chemical profiles for in vivo toxicity prediction and mechanism characterization,” Nat Commun. 7, p. 10425; and Huang et al., 2018, “Expanding biological space coverage enhances the prediction of drug adverse effects in human using in vitro activity profiles,” Sci Rep. 8(1):3783, each of which is hereby incorporated herein by reference in its entirety.

In some embodiments, the one or more cellular-components are quantified using single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, or any combinations thereof, or summaries of the same, including combinations, such as linear combinations, representing activated pathways in the single-cell cellular-component expression datasets. In some embodiments, the cellular-component measurements include gene expression measurements, such as RNA levels. The cellular-component expression measurement can be selected based on the desired cellular-component to be measured.

In some embodiments, statistical techniques are applied to quantifying cellular-components in a cell of a population of cells under the theory that varying cellular-component expression, associated with varying presence, absence or amounts of one or more measured cellular-components of interest, at different stages in cell state transition provides a high dimensional dataset from which meaningful knowledge can be extracted. In practice, the number of cellular-components may be on the order of thousands to tens of thousands, making the computations described herein impractical if not impossible to perform mentally or by hand.

In some embodiments, these statistical techniques can be characterized as methods in which the high dimensional data is compressed down to a lower dimensional space while preserving the shape of whatever latent information is encoded in the datasets. The low dimensional data is evaluated to identify differentially present cellular-components between different stages of cell state transition. Any one of a number of methods and metrics may be used to identify which of those cellular-components are sufficiently “differently” expressed relative to other cellular-components so as to be tagged as “differentially expressed” in accordance with this description. In some embodiments, the identification of cellular-components that are differentially present (e.g., differentially expressed) also provides insight into whether and/or how such cellular-components impact or associate with cell state transitions.

By matching differential cellular-component expression that characterizes a particular cellular transition to differential cellular-component expression caused by exposure of a cell to a perturbation, perturbations that affect the particular cell state transition can be predicted. A perturbation of a cell includes any treatment of the cell with one or more compounds. The one or more compounds can include, for example, a small molecule, a biologic, a protein, a protein combined with a small molecule, an ADC, a nucleic acid, such as an siRNA or interfering RNA, a cDNA over-expressing wild-type and/or mutant shRNA, a cDNA over-expressing wild-type and/or mutant guide RNA (e.g., Cas9 system or other cellular-component editing system), or any combination of any of the foregoing. Differentially expressed cellular-components for a particular cellular transition can be compared with differentially expressed cellular-components caused by exposure of a cell to a perturbation. Then, the perturbations that cause differential cellular-component expression that matches the differential cellular-component expression of the particular cellular transition can be predicted to affect the particular cellular transition. For example, in some preferred embodiments, the matching provides each perturbation (e.g., compound) with a respective one or more biological properties, including, but not limited to, cell state transitions. Such methods provide advantages over conventional techniques by associating compounds with discrete biological states while reducing the complexity, dimensionality, and potential noise of the respective characteristic profiles (e.g. when directly associating perturbations with gene expression, proteomics, and/or metabolomics profiles). Furthermore, the reduction of dimensionality further improves the performance of downstream applications such as de novo molecule generation by decreasing the computational burden and subsequently decreasing resource requirements.

In some embodiments, to predict perturbations that affect a particular cellular transition by matching differential cellular-component expression that characterizes the particular cellular transition to differential cellular-component expression caused by exposure of a cell to a perturbation, first, the most differentially expressed cellular-components that characterize the particular cellular transition are identified. In some embodiments, these differentially expressed cellular-components are identified using one of a difference of means test, a Wilcoxon rank-sum test (Mann Whitney U test), a t-test, a logistic regression, and a generalized linear model. In alternative embodiments, any statistical method may be used to identify the most differentially expressed cellular-components for a particular cellular transition. The resulting ranked table (or list) of cellular-component names and significance scores quantifies an association between a change in cellular-component expression of the cellular-component and a change in cell type between the original cell type and the transitioned cell type. In aggregate, these scores form an overall measure of the differential cellular-component expression associated with transition between the original cell type (first cell state) and the transitioned cell type (altered cell state).

Similarly, in some embodiments, differential cellular-component expression caused by exposure of a cell to a perturbation is identified for one or more perturbations. In some such embodiments, to identify differential cellular-component expression caused by exposure of a cell to a perturbation, the cellular-component expression in the cell exposed to the perturbation is compared to the cellular-component expression in control cell(s) that have not been exposed to the perturbation or an average over unrelated perturbed samples. In some embodiments, this comparison is performed using a one of difference of means test, a Wilcoxon rank-sum test (Mann Whitney U test), a t-test, a logistic regression, and a generalized linear model. In alternative embodiments, any statistical method may be used to perform the comparison. In further alternative embodiments, a statistical or machine learning model for classifying perturbations may be fitted, and its latent or output representation used for matching cellular transitions. In further alternative embodiments, the differential cellular-component expression caused by exposure of the cell to a perturbation may be known and identified from literature.

In certain embodiments, covariates of a perturbation may exist. For example, if the perturbations are small molecules, covariates of a small molecule may include, a specific dose of the small molecule, a time at which the cell exposed to the small molecule is measured to quantify cellular-components, and/or the identity (e.g., cell line) of the cell exposed to the small molecule. In some embodiments, a perturbation is predicted to affect a particular cellular transition only when a threshold quantity of its covariates are also predicted to affect the particular cellular transition. For example, a perturbation may be predicted to affect a particular cellular transition only when at least two of its covariates are also predicted to affect the particular cellular transition.

In some embodiments, alternate methods of matching are used. For example, cellular-components may be matched to a database using a web interface (See, e.g., Duan, 2016, “L1000CDS². An ultra-fast LINCS L1000 Characteristic Direction Signature Search Engine.” Systems Biology and Applications 2, article 16015, which is hereby incorporated by reference).

In some embodiments, a biological utility is identified for a perturbation. For example, measurements of one or more cellular-components (or combination of different cellular-components) can indicate differential levels or differential presence in cells having different states or phenotypes, e.g., diseased and normal phenotypes. That is, the presence, absence, or amount of cellular-component is associated with a cell state or phenotype. In some embodiments, the biological utility of a perturbation is measured by exposing a plurality of cells to a perturbation (e.g., a compound) and carrying out a first differential cellular-component expression assay, where the assay includes accessing a first plurality of single-cell expression datasets obtained from a plurality of cells prior to and following exposure of the cells to the perturbation. For example, in some embodiments, the cellular-component is a cell state or phenotype exhibited by a population of cells in a cell culture (e.g., an in vitro cell culture. In some embodiments, the cellular-component is a cell state or phenotype exhibited by a population of cells from a biological tissue (e.g., an in vitro or in vivo tissue sample). In some embodiments, the cellular-component is a cell state or phenotype exhibited by one or more subsets of the population of cells (e.g., a healthy or an unhealthy sub-population of cells).

In some embodiments, the plurality of cells comprises at least 10, at least 50, at least 100, at least 200, at least 300, at least 400, at least 500, at least 600, at least 700, at least 800, at least 900, or at least 1000 cells. In some embodiments, the plurality of cells comprises at least 100, at least 1000, at least 5000, at least 1×10⁴, at least 2×10⁴, at least 3×10⁴, at least 4×10⁴, at least 5×10⁴, at least 6×10⁴, at least 7×10⁴, at least 8×10⁴, at least 9×10⁴, at least 1×10, at least 2×10⁵, at least 3×10⁵, at least 4×10⁵, at least 5×10⁵, at least 6×10⁵, at least 7×10⁵, at least 8×10⁵, at least 9×10⁵, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 2×10⁷, at least 3×10⁷, or at least 5×10⁷ cells. In some embodiments, the plurality of cells comprises no more than 10, no more than 50, no more than 100, no more than 200, no more than 300, no more than 400, no more than 500, no more than 600, no more than 700, no more than 800, no more than 900, or no more than 1000 cells. In some embodiments, the plurality of cells comprises no more than 100, no more than 1000, no more than 5000, no more than 1×10⁴, no more than 2×10⁴, no more than 3×10⁴, no more than 4×10⁴, no more than 5×10⁴, no more than 6×10⁴, no more than 7×10⁴, no more than 8×10⁴, no more than 9×10⁴, no more than 1×10⁵, no more than 2×10⁵, no more than 3×10⁵, no more than 4×10⁵, no more than 5×10⁵, no more than 6×10⁵, no more than 7×10⁵, no more than 8×10⁵, no more than 9×10⁵, no more than 1×10⁶, no more than 2×10⁶, no more than 3×10⁶, no more than 4×10⁶, no more than 5×10⁶, no more than 6×10⁶, no more than 7×10⁶, no more than 8×10⁶, no more than 9×10⁶, no more than 1×10⁷, no more than 2×10⁷, no more than 3×10⁷, or no more than 5×10⁷ cells. In some embodiments, the plurality of cells comprises between 1 and 10, between 10 and 100, between 100 and 1000, between 1000 and 1×10⁴, between 1×10⁵ and 1×10⁶, between 1×10⁶ and 1×10⁷, or more than 1×10⁷ cells.

As another example, in some embodiments, a population of cells includes two sub-populations of cells, including one healthy sub-population and one unhealthy (e.g., diseased) sub-population. During cell culturing, a plurality of different perturbations may be introduced into the unhealthy sub-population. Through subsequent single-cell expression measurement in conjunction with the methods described herein, it can be determined what effect the perturbations had in the differential cellular-component expression of the cellular-components in the unhealthy sub-population, particularly in related to the healthy sub-population. For example, a subset of the cells from the un-healthy sub-population exposed to one or more perturbations may exhibit cellular-component expression consistent with the healthy sub-population of cells, indicating that the perturbation had a desirable effect on the un-healthy sub-population of cells.

Additionally, different subsets of the population of cells may be perturbed in different ways beyond simply mixing many perturbations and post-hoc evaluating which cells were affected by which perturbations. For example, if the population of cells is physically divided into different wells of a multi-well plate, then different perturbations may be applied to each well. Other ways of accomplishing different perturbations for different cells are also possible.

In some embodiments, the diseased cell phenotype is identified by a discrepancy between the diseased cell and a normal cell. For instance, in some embodiments, the diseased cell phenotype can be identified by loss of a function of the cell, gain of a function of the cell, progression of the cell (e.g., transition of the cell into a differentiated state), stasis of the cell (e.g., inability of the cell to transition into a differentiated state), intrusion of the cell (e.g., emergence of the cell in an abnormal location), disappearance of the cell (e.g., absence of the cell in a location where the cell is normally present), disorder of the cell (e.g., a structural, morphological, and/or spatial change within and/or around the cell), loss of network of the cell (e.g., a change in the cell that eliminates normal effects in progeny cells or cells downstream of the cell), a gain of network of the cell (e.g., a change in the cell that triggers new downstream effects in progeny cells of cells downstream of the cell), a surplus of the cell (e.g., an overabundance of the cell), a deficit of the cell (e.g., a density of the cell being below a critical threshold, a difference in cellular-component ratio and/or quantity in the cell, a difference in the rate of transitions in the cell, or any combination thereof.

In some embodiments, the diseased cells include cell lines, biopsy sample cells, and cultured primary cells. In some embodiments, the normal cells include cultured primary cells and biopsy sample cells. In some embodiments, the cells are human cells.

In some embodiments, the methods are used to select a perturbation (e.g., compound) useful for treating a disease, based on an indicated utility identified using the above-described methods. In some embodiments, the methods include treating a subject having a disease by administering to the subject an effective amount of a selected perturbation or a drug substance developed from a perturbation lead compound. In some embodiments, the perturbation (e.g., compound) is known to have an acceptable human safety profile determined by results obtained in a regulated clinical trial.

Further details relating to dimensionality reduction to identify differentially expressed cellular components and/or matching perturbations to cell states and/or cell state transitions are discussed in International Patent Application No. PCT/US2019/041976, entitled “Methods of Analyzing Cells,” filed Jul. 16, 2019, which is hereby incorporated herein by reference in its entirety.

Featurization

Referring to Block 206, the disclosed method further comprises training an untrained or partially untrained neural network encoder and an untrained or partially untrained classifier by performing a first procedure. The first procedure comprises, for each respective compound in the first plurality of compounds, projecting the information regarding the chemical structure of the respective compound into a latent representation space in accordance with a first plurality of weights associated with the untrained or partially untrained neural network encoder to obtain a corresponding projected representation of the respective compound. The corresponding projected representation of the respective compound is inputted into the untrained or partially untrained classifier to obtain a classification of the respective compound in accordance with a second plurality of weights associated with the untrained or partially untrained classifier. The first plurality of weights and the second plurality of weights are updated by comparing the classification of each respective compound in the first plurality of compounds to the one or more biological properties of the respective compound in the first training dataset, thus obtaining a trained neural network encoder and a trained classifier.

In some embodiments, the first training dataset is obtained by removing (e.g., holding out) a subset of compounds from a first plurality of compounds, and the removed subset of compounds from the first plurality of compounds is used to verify that the trained neural network encoder and the trained classifier correctly classifies a respective compound from the removed subset of compounds.

In some embodiments, the corresponding projected representation has N-dimensions. In some such embodiments, N is an integer between 20 and 80. In some embodiments, N is 50. In some embodiments, N is an integer between 2 and 10, between 10 and 20, between 20 and 30, between 30 and 40, between 40 and 50, between 50 and 60, between 60 and 70, between 70 and 80, between 80 and 90, or between 90 and 100. In some embodiments. N is at least 20, at least 30, at least 40, at least 50, at least 60, at least 70, at least 80, at least 90, at least 100, at least 120, at least 140, at least 160, at least 180, at least 200, at least 300, at least 400, or at least 500. In some embodiments, N is an integer between 2 and 2000, between 5 and 1500, between 10 and 1000, or between 20 and 500.

In some embodiments, the information regarding the chemical structure of the respective compound is a molecular structure of the respective compound, and the method further comprises forming a featurization of the chemical structure and incorporating the featurization of the chemical structure into a multi-dimensional vector space. Projecting the information regarding the chemical structure of the respective compound into the latent representation space in accordance with the first plurality of weights associated with the untrained or partially untrained neural network encoder comprises inputting the multi-dimensional vector space of the chemical structure into the untrained or partially untrained neural network encoder.

Specifically, in some embodiments, the first step of the training phase is the featurization of the molecule structure.

In some embodiments, the goal of featurization is to convert molecules into tensors such that they can be processed (e.g., by parametric algebraic operations). Thus, in some embodiments, the featurization of the chemical structure is a tensor. In some such embodiments, the tensor is a one-dimensional vector or a two-dimensional matrix.

There are several ways to featurize a molecule. In some embodiments, the featurization of the chemical structure is an extended circular fingerprint (e.g., ECF or Morgan), or a molecular graph of a plurality of one-hot-encoded vectors. This is calculated by first defining a list of atoms that can be found in an organic molecule, then representing each atom in a molecule by an array where all entries are zero except the one that corresponds to the index of the atom of interest. This list of one-hot encoded vectors is accompanied by an adjacency matrix which informs about the connectivity between atom pairs in the molecule structure. Methods for one-hot encoding are known in the art, as described, for example, in Brownlee, 2017, “Why One-Hot Encode Data in Machine Learning?” Machine Learning Mastery, available online at machinelearningmastery.com/why-one-hot-encode-data-in-machine-learning; and Brownlee, 2020, “Ordinal and One-Hot Encodings for Categorical Data,” Machine Learning Mastery, available online at machinelearningmastery.com/one-hot-encoding-for-categorical-data, both of which is hereby incorporated herein by reference in its entirety.

In some embodiments, the forming the featurization of the chemical structure comprises converting the chemical structure to a simplified molecular-input line-entry system (SMILES) string, and converting the SMILES string into a molecular graph representation that comprises an adjacency matrix and a feature matrix. Methods for conversion of chemical structures to SMILES strings are described in, for example, Weininger, 1988, “SMILES, a chemical language and information system. 1. Introduction to methodology and encoding rules,” J Chem Inf and Comp Sci, 28(1): 31-6; doi:10.1021/ci00057a005, which is hereby incorporated herein by reference in its entirety.

Molecular Structure Encoder

After the featurization step, the molecules (e.g., their chemical structure and/or the featurization of their chemical structure) are encoded into a high-dimensional (e.g., multi-dimensional) vector space, where the dimension can be large enough to represent the rich information about the molecules' relevant physio-chemical properties. Such encoding is performed by a series of algebraic operations whose parameters are to be learned in an optimization process (e.g., the embedding step of the training phase).

In some embodiments, the incorporating the featurization of the chemical structure into the multi-dimensional vector space for the chemical structure comprises inputting the featurization of the chemical structure into a spatial graph convolutional network (GCN). In some embodiments, the GCN is a graph attention network (GAT), a graph isomorphism network (GIN), or a graph substructure index-based approximate graph (SAGA).

For example, for each variant in the plurality of variants of the spatial graph convolution network (GCN), a plurality of layers can be used such that each respective atom in a plurality of individual atomic feature representations are updated with new properties that come from neighboring atoms at each layer. Therefore, for example, stacking up 5 GCN layers informs each respective atom from 5^{th} degree connections. In some such embodiments, an aggregation operation (e.g., a mean or sum) is applied to all of the updated vectors that correspond to neighbors of each atom.

In some embodiments, the incorporating the featurization of the molecular structure into the multi-dimensional vector space for the chemical structure comprises an application of a spectral graph convolution (SGC) to the featurization of the chemical structure. In some embodiments, the application of the SGC to the featurization of the chemical structure uses Chebyshev polynomial filtering (see, for example, Defferrard et al., 2016, “Convolutional Neural Networks on Graphs with Fast Localized Spectral Filtering,” NIPS Advances in Neural Information Processing Systems 29. arXiv: 1606.09375, which is hereby incorporated herein by reference in its entirety).

For example, the spectral graph convolution method differs from the spatial convolution method in that the adjacency matrix representing the atomic graph is first converted into its Laplacian, where the Laplacian of a graph can be considered as a normalized adjacency matrix. The Eigen decomposition of the Laplacian provides its spectrum and constructs an orthogonal basis of the operator. The convolution theorem states that a convolution in the spatial domain corresponds to the multiplication in the corresponding adjoint spectral domain. One layer of spectral graph convolution is defined such that the result of matrix multiplication of transposed eigen vectors and the feature vectors is elementwise multiplied with the result of matrix multiplication of transposed eigen vectors and spectral filters, followed by the matrix multiplication by eigen vectors, resulting in the updated feature vectors:

X^{(l+1)}=V(V^{T}X^{(l)}⊙V^{T}W^{(l)} (Eq. 1)

where X^{l} is the feature vector of layer l. V is the eigen vector matrix, and W is the spectral filter matrix. In the naïve implementation, the spectral filters (W) are as large as the graph size and cannot efficiently represent the recurring small patterns in the graph. For example, two benzene rings that are attached to the same backbone will be represented separately. To alleviate this issue, the spectral filters may, in some embodiments, be represented as the weighted combination of smooth functions, where the weights are the parameters to be learned during the training phase and have much smaller dimension than the original size of the graph, thus regularizing potentially highly irregular weight matrices and enforcing patterns that will display properties of spatial translation:

$${W}^{( l )} ≅ {\sum }_{k = 1}^{K} {α}_{k} {f}_{k} \qquad ( Eq . 2 )$$

In Equation 2, K is a number that should intuitively correspond to number of functional groups in a molecule which is less than N (e.g., the number of atoms in a graph). In some embodiments, Chebyshev polynomials are used as a smooth function to construct the spectral filter. In some embodiments, K is 3. In some alternative embodiments, K is greater than 3.

In some embodiments, similar performance is attained using either spatial convolution methods and/or spectral convolution methods. In some embodiments, the encoding is performed using any one of possible options and variants for encoding that will be apparent to one skilled in the art.

In some embodiments, the multi-dimensional vector space is an N-dimensional space, wherein N is an integer between 20 and 80. In some embodiments, N is 50. In some embodiments, N is an integer between 2 and 10, between 10 and 20, between 20 and 30, between 30 and 40, between 40 and 50, between 50 and 60, between 60 and 70, between 70 and 80, between 80 and 90, or between 90 and 100. In some embodiments, N is at least 20, at least 30, at least 40, at least 50, at least 60, at least 70, at least 80, at least 90, at least 100, at least 120, at least 140, at least 160, at least 180, at least 200, at least 300, at least 400, or at least 500. In some embodiments, N is between 2 and 2000.

Constrained Representation Learning

In some embodiments, after embedding the molecule structure into a high dimensional vector space, one or more constraints for high-level design criteria are provided and the corresponding projected representation of the respective compound is optimized such that the representation satisfies these constraints (e.g., the constrained representation step of the training phase). In some embodiments, such constraints vary across multiple scales and/or biological states (e.g., agonizing or antagonizing a particular kinase or other protein class, upregulating or inhibiting a particular pathway, and/or promoting or blocking a particular cellular transition).

In some embodiments, the one or more constraints comprises 1, 2, 3, 4, 5, 6, 7, 8, 9, 10, 11, 12, 13, 14, 15, 16, 17, 18, 19, 20, or more than 20 constraints. In some embodiments, the plurality of constraints comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 constraints. In some embodiments, the plurality of biological properties comprises between 1 and 5, between 5 and 10, between 10 and 20, between 20 and 30, between 30 and 40, between 40 and 50, or between 50 and 100 constraints.

In some embodiments, the constrained representation learning is performed using a classifier such as, for example, a logistic regression classifier, a k-nearest neighbor classifier, a deep neural network classifier, a support vector machine classifier, a decision tree classifier, or a naïve Bayes classifier, etc.

Logistic regression classifiers are disclosed in Agresti, An Introduction to Categorical Data Analysis, 1996, Chapter 5, pp. 103-144, John Wiley & Son, New York, which is hereby incorporated by reference. In some embodiments, the logistic regression classifier includes at least 10, at least 20, at least 50, at least 100 weights, or at least 1000 weights and requires a computer to calculate because it cannot be mentally solved.

A k-nearest neighbor classifier is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See. Duda et al., 2001. Pattern Classification, Second Edition, John Wiley & Sons, which is hereby incorporated by reference. In some embodiments, the number of distance calculations needed to solve the k-nearest neighbor classifier is such that a computer is used to solve the classifier for a given input because it cannot be mentally performed.

A deep neural network classifier comprises an input layer, a plurality of individually weighted convolutional layers, and an output scorer. The weights of each of the convolutional layers as well as the input layer contribute to the plurality of weights associated with the deep neural network classifier. In some embodiments, at least 100 weights, at least 1000 weights, at least 2000 weights or at least 5000 weights are associated with the deep neural network classifier. As such, deep neural network classifiers require a computer to be used because they cannot be mentally solved. In other words, given an input to the classifier, the classifier output needs to be determined using a computer rather than mentally in such embodiments. See, for example, Krizhevsky et al., 2012, “Imagenet classification with deep convolutional neural networks,” in Advances in Neural Information Processing Systems 2, Pereira. Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, Mass., USA: MIT Press, each of which is hereby incorporated by reference.

SVM classifiers are described in Cristianini and Shawe-Taylor, 2000. “An Introduction to Support Vector Machines,” Cambridge University Press, Cambridge: Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor. N.Y.; Duda, Pattern Classification. Second Edition. 2001, John Wiley & Sons, Inc., pp. 259, 262-265; and Hastie, 2001. The Elements of Statistical Learning, Springer, New York; and Furey et al., 2000. Bioinformatics 16, 906-914, each of which is hereby incorporated by reference in its entirety. When used for classification, SVMs separate a given set of binary labeled data training set with a hyper-plane that is maximally distant from the labeled data. For cases in which no linear separation is possible, SVMs can work in combination with the technique of “kernels,” which automatically realizes a non-linear mapping to a feature space. The hyper-plane found by the SVM in feature space corresponds to a non-linear decision boundary in the input space. In some embodiments, the plurality of weights associated with the SVM define the hyper-plane. In some embodiments, the hyper-plane is defined by at least 10, at least 20, at least 50, or at least 100 weights and the SVM classifier requires a computer to calculate because it cannot be mentally solved.

Decision tree classifiers are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda. 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests-Random Features,” Technical Report 567, Statistics Department, U.C. Berkeley, September 1999, which is hereby incorporated by reference in its entirety. In some embodiments, the decision tree classifier includes at least 10, at least 20, at least 50, or at least 100 weights (decisions) and requires a computer to calculate because it cannot be mentally solved.

Naïve Bayes classifiers. A Naive Bayes classifier is any classifier in a family of “probabilistic classifiers” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

In some simplified embodiments, a projected representation is optimized using a softmax classifier, such that representations corresponding to cell states and/or biological states can be classified. In some embodiments, the constraints are implemented by requiring proximity (e.g., as measured by a common vector space metric such as Euclidean distance) between molecules that belong to the same constraint class (e.g., induction of a specific pathway and/or cell state) in a projected subspace (e.g., subspaces preceding a softmax classifier).

For example, molecules that prolong cell cycle can have a wide variety of molecular structures, which can appear scattered in the original feature space. However, when their original feature vectors are processed using one of the graph-based encoders, the vectors corresponding to the molecules that share the same high-level property (e.g., constraint class) are located in close proximity to each other in some standard metric of the latent vector space. If multiple constraints are provided at the same time (e.g., using multi-task learning), the embedded representations are projected into subspaces such that the proximity objective holds in each subspace separately. Thus, in some embodiments, a molecular embedding space can comprise many different projections that satisfy many different constraints (e.g., liver toxicity, cell state change) for which the molecular targets can be elucidated. In some embodiments, each projection satisfies a single constraint (e.g., liver toxicity, cell state change, etc.). In some embodiments, each projection satisfies 2, 3, 4, 5, 6, 7, 8, 9, 10, 11, 12, 13, 14, 15, 16, 17, 18, 19, 20, or more than 20 different constraints.

In some embodiments, a constraint corresponds to a biological property of a respective molecule (e.g., compound). In some embodiments, a constraint is a biological property measured via a compound activity assay. For example, in some embodiments, a constraint is the compound activity of a respective molecule, determined based upon an estrogen receptor alpha (ER-alpha) compound screening assay and/or an auto-fluorescence counter screen, where the auto-fluorescence counter screen is performed as a proxy for toxicity-dependent cell death. In some embodiments, a constraint is the compound activity of a respective molecule, determined based upon an aryl hydrocarbon receptor (AhR) antagonist mode assay and/or a cell viability counter screen. In some embodiments, a constraint is the compound activity of a respective molecule, determined based upon an estrogen receptor alpha (ER-alpha) compound screening assay, an aryl hydrocarbon receptor (AhR) antagonist mode assay, an aromatase antagonist mode assay, an androgen receptor (AR) assay, peroxisome proliferator-activated receptor gamma (PPAR-gamma) agonist mode assay, a nuclear factor (erythroid-derived 2)-like 2/antioxidant responsive element (Nrf2/ARE) mode assay, a heat shock factor response element (HSE) mode assay, an ATAD5 mode assay, a mitochondrial membrane potential (MMP), a p53 mode assay, a cell viability counter screen, and/or an auto-fluorescence counter screen. Further assays for selecting and/or determining constraints used for generating representations are contemplated, as are described in Huang et al. 2016, “Modelling the Tox21 10 K chemical profiles for in vivo toxicity prediction and mechanism characterization,” Nat Commun. 7, p. 10425, which is hereby incorporated by reference.

In some embodiments, a constraint is a biological property shared between two or more molecules in a plurality of molecules. In some embodiments, a constraint is a biological property shared between 2, 3, 4, 5, 6, 7, 8, 9, 10, 11, 12, 13, 14, 15, 16, 17, 18, 19, 20, or more than 20 molecules. In some embodiments, a constraint is a biological property shared between at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 molecules. In some embodiments, a constraint is a biological property shared between at least 1000, at least 2000, at least 3000, at least 4000, at least 5000, at least 6000, at least 7000, at least 8000, at least 9000, or at least 10000 molecules.

In some embodiments, the biological property is measured in one or more cell lines, including human cell lines, animal (e.g., hamster, chicken, rat, and/or mouse) cell lines, and/or one or more tissue types (e.g., liver, kidney, ovarian, cervical cancer, breast cancer, and/or colon cancer). In some embodiments, the biological property is measured in a healthy cell line and/or an unhealthy cell line (e.g., a cancerous cell line). In some embodiments, a cell line is selected from the group consisting of HepG2, ME-180, HEK293, MDA-MB-453, MCF-7, CHO, DT40, BG1, HeLa, GH3, HCT-116, C3H10T1/2, and NIH/3T3. In some embodiments, the biological property is measured in at least 1, at least 2, at least 3, at least 4, at least 5, at least 6, at least 7, at least 8, at least 9, or at least 10 cell lines. In some embodiments, the biological property is measured using any of the methods or embodiments described in Huang R, 2016, “A Quantitative High-Throughput Screening Data Analysis Pipeline for Activity Profiling,” High-Throughput Screening Assays in Toxicology, Methods in Molecular Biology; 1473(1); Huang el al., 2016, “Modelling the Tox21 10 K chemical profiles for in vivo toxicity prediction and mechanism characterization,” Nat Commun. 7, p. 10425; and Huang et al., 2018, “Expanding biological space coverage enhances the prediction of drug adverse effects in human using in vitro activity profiles,” Sci Rep. 8(1).3783, each of which is hereby incorporated herein by reference in its entirety, and/or any substitutions, additions, deletions, modifications, and/or combinations thereof as will be apparent to one skilled in the art.

In some embodiments, the training of the neural network encoder (e.g., projecting the information regarding the chemical structure of the respective compound into the latent representation space) and the training of the classifier (e.g., inputting the projected representation of the respective compound) is performed using a plurality of compounds in the first training dataset comprising information regarding a single biological and/or functional pathway.

In some alternative embodiments, the training of the neural network encoder and the training of the classifier is performed using multi-task learning, where a plurality of compounds in the first training dataset comprising information regarding a plurality of biological and/or functional pathways is inputted into the neural network encoder and the classifier. Due to the co-activation of multiple biological pathways and/or the increased coverage of the one or more compounds that induce multiple biological states, in some such embodiments, multi-task learning increases the accuracy and robustness of classification by providing information on biological pathway interconnectivity.

In some embodiments, the trained neural network encoder and the trained classifier comprise an updated first plurality of weights associated with the trained neural network encoder and an updated second plurality of weights associated with the trained classifier. In some embodiments, the first plurality of weights comprises 10, 20, 50, 100, 500, 1000, 5000, or 10,000 or more weights. In some embodiments, the second plurality of weights comprises 10, 20, 50, 100, 500, 1000, 5000, or 10,000 or more weights. In some embodiments, the updating of the first and second plurality of weights is performed using back-propagation. For example, in some embodiments of machine learning (e.g., deep learning), back-propagation is a method of training a network with hidden layers comprising a plurality of weights. The output of the untrained or partially untrained neural network encoder and the untrained or partially untrained classifier using the initial weights (e.g., the classification of the respective compound in accordance with the first and second plurality of weights) is compared with the actual classification (e.g., the first biological property of the respective compound) and the error is computed (e.g., using a loss function). The weight values are then updated such that the error is minimized (e.g., according to the loss function). In some embodiments, any one of a variety of back-propagation algorithms and/or methods are used to update the first and second plurality of weights, as will be apparent to one skilled in the art. In an exemplary embodiment, the neural network is trained against the errors in class assignment made by the network, in view of the training data, by stochastic gradient descent with the AdaDelta adaptive learning method (Zeiler, 2012 “ADADELTA: an adaptive learning rate method,” CoRR, vol. abs/1212.5701, which is hereby incorporated by reference), and the back-propagation algorithm provided in Rumelhart et al., 1988, “Neurocomputing: Foundations of research,” ch. Learning Representations by Back-propagating Errors, pp. 696-699, Cambridge, Mass., USA: MIT Press, which is hereby incorporated by reference.

In some embodiments, the updated first and second plurality of weights encodes each respective compound in the first plurality of compounds such that each the projected representation of each respective compound in the first plurality of compounds forms a cluster corresponding to one or more functionally enriched groups (e.g., a biological and/or functional pathway, a cell state or biological state, and/or a cell state or biological state transition). In some embodiments, latent representations of cell state activations can be visualized using multi-dimensional scaling algorithms (e.g., NuMap) and/or 2-dimensional prediction algorithms (e.g., t-distributed stochastic neighbor embedding, disclosed for example in van der Maaten, 2008, “Visualizing Data Using t-SNE,” Journal of Machine Learning Research 9: 2579-2605, which is hereby incorporated by reference).

Molecule Generation

Referring to Block 208, the method further comprises obtaining a second training dataset, in electronic form. The second training dataset comprises, for each respective compound in a second plurality of compounds (e.g., comprising 100 or more compounds), information regarding a chemical structure of the respective compound.

Referring to Block 210, the method further comprises training an untrained or partially untrained decoder by performing a second procedure. The second procedure comprises, for each respective compound in the second plurality of compounds, projecting the information regarding the chemical structure of the respective compound into a latent representation space in accordance with the first plurality of weights associated with the trained neural network encoder to obtain a corresponding projected representation of the respective compound, and inputting the corresponding projected representation of the respective compound into the untrained or partially untrained decoder to obtain a chemical structure of the respective compound in accordance with a third plurality of weights associated with the untrained or partially untrained decoder. The third plurality of weights is updated by comparing the chemical structure of each respective compound outputted by the untrained or partially untrained decoder to the actual chemical structure of the respective compound from the second training dataset thereby obtaining a trained decoder.

In some embodiments, the updating of the third plurality of weights is performed using back-propagation as described above. In some such embodiments, the output of the untrained or partially untrained decoder using the initial weights (e.g., the chemical structure of the respective compound outputted in accordance with the third plurality of weights) is compared with the actual chemical structure and the error is computed (e.g., using a loss function) such that the error can be minimized.

In some embodiments, the second training dataset is the same as the first training dataset. In some embodiments, the second training dataset is obtained by removing (e.g., holding out) a subset of compounds from a second plurality of compounds, and the removed subset of compounds from the second plurality of compounds is used to verify that the trained decoder reconstructs the chemical structure of a respective compound from the removed subset of compounds.

In some embodiments, the second training dataset comprises virtual compounds. In some embodiments, the second training dataset is a small molecules and/or ligand dataset. In some embodiments, the second training dataset is all or a portion of a ZINC dataset. See, for example, Irwin and Shoichet, “ZINC—A Free Database of Commercially Available Compounds for Virtual Screening,” J Chem Inf Model. 2005: 45(1): 177-182, which is hereby incorporated herein by reference in its entirety.

In some embodiments, the second training dataset comprises 100 or more, 1,000 or more, 10,000 or more, 100,000 or more, 250,000 or more, 500,000 or more, 1 million or more, 2 million or more, or 5 million or more compounds. In some embodiments, the second training dataset does not include functional data (e.g., one or more biological properties). In some embodiments, the second plurality of compounds comprises at least 10, at least 20, at least 25, at least 30, at least 35, at least 40, at least 45, at least 50, at least 55, at least 60, at least 65, at least 70, at least 75, at least 80, at least 85, at least 90, at least 95, or at least 100 compounds. In some embodiments, the second plurality of compounds comprises at least 50, at least 100, at least 200, at least 300, at least 400, at least 500, at least 600, at least 700, at least 800, at least 900, or at least 1000 compounds. In some embodiments, the second plurality of compounds comprises at least 1000, at least 2000, at least 3000, at least 4000, at least 5000, at least 10,000, at least 100,000, or at least 1 million compounds. In some embodiments, the second plurality of compounds comprises no more than 10, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 compounds. In some embodiments, the second plurality of compounds comprises no more than 50, no more than 100, no more than 200, no more than 300, no more than 400, no more than 500, no more than 600, no more than 700, no more than 800, no more than 900, or no more than 1000 compounds. In some embodiments, the second plurality of compounds comprises no more than 1000, no more than 2000, no more than 3000, no more than 4000, no more than 5000, no more than 10,000, no more than 100,000, no more than 1 million, no more than 2 million, no more than 5 million, or no more than 10 million compounds. In some embodiments, the second plurality of compounds comprises between 2 and 20, between 20 and 50, between 50 and 100, between 100 and 200, between 200 and 500, between 500 and 1000, between 1000 and 5000, between 5000 and 10,000, between 10,000 and 100,000, between 100,000 and 1 million, or between 1 million and 5 million compounds.

In some embodiments, the projected representation is obtained using any of the methods disclosed herein. In some embodiments, the corresponding projected representation has N-dimensions. In some such embodiments, N is an integer between 20 and 80. In some embodiments, N is 50. In some embodiments, N is an integer between 2 and 10, between 10 and 20, between 20 and 30, between 30 and 40, between 40 and 50, between 50 and 60, between 60 and 70, between 70 and 80, between 80 and 90, or between 90 and 100. In some embodiments, N is at least 20, at least 30, at least 40, at least 50, at least 60, at least 70, at least 80, at least 90, at least 100, at least 120, at least 140, at least 160, at least 180, at least 200, at least 300, at least 400, or at least 500. In some embodiments, N is an integer between 2 and 2000, between 5 and 1500, between 10 and 1000, or between 20 and 500.

Methods of Use

Referring to Block 212, the method further comprises using the trained neural network encoder, trained classifier, and trained decoder to identify a test compound that has the first biological property, where the test compound is not present in the first and second training set.

In some embodiments, the trained neural network encoder, trained classifier, and trained decoder are verified by a third procedure. A first compound is obtained, not present in the first or second training dataset, that has the first biological property and has a known chemical structure. A projected representation for the first compound is obtained by inputting a chemical structure of the first compound into the trained neural network encoder. The projected representation of the first compound is inputted into the trained classifier to verify that the trained classifier identifies the first compound as having the first biological property. The projected representation of the first compound is inputted into the trained decoder to verify that the trained decoder reconstructs the chemical structure of the first compound.

In some such embodiments, the verification (e.g., validation) is performed using a “hold-one-out” method, where one or more compounds from the first or second training dataset is removed from the respective plurality of compounds in the first or second training set. The obtaining of the projected representation and subsequent verification of the trained classifier and the trained decoder is performed using the one or more compounds held out from the original first or second training datasets. In some embodiments, a 5%. 10%, 15%, 20%, or more than 20% of a training dataset is held out. In some exemplary embodiments, 600 compounds are held out of a training dataset comprising 10,600 compounds. In some embodiments, the verification is performed in silico.

One aspect of the present disclosure provides a method of discovering a test compound that has a first biological property, the method comprising using a trained neural network encoder, trained classifier, and trained decoder to identify a test compound that has a first biological property, where the trained neural network encoder, trained classifier, and trained decoder were trained by processes comprising any of the methods and embodiments disclosed above, and where the test compound is not present in the first and second training set.

For example, one aspect of the present disclosure provides a method of discovering a candidate compound that has a first biological property, the method comprising obtaining a first projected representation of a first compound that is assigned the first biological property by inputting a chemical structure of the first compound into a trained neural network encoder (e.g., where the first projected representation has N dimensions, and where N is an integer between 20 and 80). The first projection is used to obtain one or more candidate projections. Each candidate projection in the one or more candidate projections is inputted into a trained decoder thereby obtaining a plurality of candidate compounds, where the first compound is not present in the plurality of candidate compounds. For each respective candidate compound in the plurality of candidate compounds a corresponding projected representation (e.g., an N-dimensional projected representation) for the respective candidate compound is obtained by inputting a chemical structure of the candidate compound into the trained neural network encoder. A classification of the respective candidate compound is obtained by inputting the corresponding projected representation of the respective candidate compound into the trained classifier. When the trained classifier indicates that the corresponding projected representation of the respective candidate compound has the first biological property, the respective candidate compound is deemed to have the first biological property.

In some embodiments, the obtaining the one or more candidate projections is performed by sampling vectors (e.g., high-dimensional vectors) from the projected representation, such as a high-dimensional (e.g., multi-dimensional) representation space. In some such embodiments, the molecular features (e.g., information regarding a chemical structure) are inferred from the vectors that are sampled from the high-dimensional constrained representation space (e.g., by inputting the vectors into the trained decoder).

For example, in some embodiments, the sampling operation is done by adding Gaussian noise to an existing molecule representation, which is known to satisfy the constraints (e.g., the desired biological property or properties for classification). The one or more obtained vectors are fed through a variant of a recurrent neural network (RNN) as the initial latent state. The RNN variant can be a long-short term memory (LSTM) or gated recurrent unit (GRU) network, which are trained on SMILES string with an autoregression strategy (e.g., given the initial vector and the past characters, predict the next character). Once trained, at the inference time the model generates hundreds of SMILES string per second. In some embodiments, the generated SMILES strings are further filtered by checking their validity (e.g., using RDKIT). In some embodiments, the decoder (e.g., generator) is implemented using a variety of architectures that will be apparent to one skilled in the art.

In some embodiments, the first projection is used to obtain one or more candidate projections, and a classification of each respective candidate projection in the one or more candidate projections is obtained first, prior to inputting each candidate projection that has the first biological property into the trained decoder, thus obtaining one or more novel compounds that have the first biological property.

In some embodiments, a projected representation (e.g., a first projected representation, second projected representation, and/or any one or more candidate projections) has N-dimensions. In some such embodiments, N is an integer between 20 and 80. In some embodiments, N is 50. In some embodiments, N is an integer between 2 and 10, between 10 and 20, between 20 and 30, between 30 and 40, between 40 and 50, between 50 and 60, between 60 and 70, between 70 and 80, between 80 and 90, or between 90 and 100. In some embodiments, N is at least 20, at least 30, at least 40, at least 50, at least 60, at least 70, at least 80, at least 90, at least 100, at least 120, at least 140, at least 160, at least 180, at least 200, at least 300, at least 400, or at least 500. In some embodiments, N is an integer between 2 and 2000, between 5 and 1500, between 10 and 1000, or between 20 and 500.

In some alternative embodiments, the method further comprises obtaining a second projected representation of a second compound that has the biological property by inputting a chemical structure of the second compound into the trained neural network encoder. The using the first projection to obtain one or more candidate projections comprises interpolating the first projection and the second projection thereby obtaining the one or more candidate projections.

Specifically, referring to Block 214, using the trained neural network encoder, trained classifier, and trained decoder comprises interpolating a projected representation of a first compound and a projected representation of a second compound, produced by the trained neural network encoder, where the first and second compound have the first molecular property (e.g., biological property), thereby obtaining an interpolated projection. The interpolated projection is inputted into the trained decoder, thus obtaining a plurality of candidate compounds. For each respective candidate compound in all or a portion of the plurality of candidate compounds, a corresponding projected representation for the respective candidate compound is obtained by inputting a chemical structure of the candidate compound into the trained neural network encoder. A classification of the respective candidate compound is obtained by inputting the corresponding projected representation of the respective candidate compound into the trained classifier, where, when the trained classifier indicates that the corresponding projected representation of the respective candidate compound has the first biological property, the respective candidate compound is deemed to have the first biological property.

In some embodiments, the interpolation of a projected representation of a first compound and a projected representation of a second compound is performed using linear interpolation. For example, where the respective projected representations of a first and second compound are represented as data points in a multi-dimensional space, a linear interpolation is a method of curve-fitting using linear polynomials to construct new data points between the data points corresponding to the first and second compound, in each respective dimension in the multi-dimensional space. A discrete number of new data points can be constructed for each interpolation; for example, in some embodiments, the discrete number of new data points (e.g., new candidate representations) between the projected representations of the first and second compounds is 2 or more, 10 or more, 50 or more, 100 or more, 500 or more, 1000 or more, 2000 or more, 5000 or more, or more than 10,000. In some embodiments, each new candidate representation is inputted into the trained decoder to obtain the plurality of candidate compounds, where the first and second compounds are not present in the plurality of candidate compounds.

In some embodiments, the interpolation of the first and second projections is used to obtain one or more candidate projections, and a classification of each respective candidate projection in the one or more candidate projections is obtained first, prior to inputting each candidate projection that has the first biological property into the trained decoder, thus obtaining one or more novel compounds that have the first biological property.

In some embodiments, using the trained neural network encoder, trained classifier, and trained decoder comprises interpolating the projected representations of three of more compounds. In some such embodiments, the method comprises creating a smooth function over a distribution (e.g., a Gaussian mixture model) to obtain a probability distribution over a plurality of sets of compounds, such that the sampling of vectors from the high-dimensional space is performed using the probability distribution.

In some embodiments, using the trained neural network encoder, trained classifier, and trained decoder comprises identifying the center of a cluster of projected representations encoded according to the updated first plurality of weights associated with the neural network encoder (e.g., visualized using t-SNE). In some such embodiments, the center of each cluster comprises the one or more candidate projections that is inputted into the decoder, thereby identifying one or more candidate compounds. In some embodiments, the using the trained neural network encoder, trained classifier, and trained decoder comprises using a first one or more candidate projections, obtained by identifying the center of a cluster of projected representations, to obtain a second one or more candidate projections using a sampling method for the first one or more candidate projections (e.g., an interpolation, Gaussian distribution, and/or probability distribution).

In some embodiments, using the trained neural network encoder, trained classifier, and trained decoder comprises obtaining a first one or more projected representations by inputting a vector of random noise.

In some embodiments, each respective candidate compound in the plurality of candidate compounds is different from any other candidate compound in the plurality of candidate compounds. In some embodiments, one or more respective candidate compounds in the plurality of candidate compounds is the same.

In some embodiments, one or more identified candidate compounds comprise a novel structure with an unknown function (e.g., with respect to clinical effect). In some embodiments, one or more identified candidate compounds comprise a known (e.g., commercially available) structure with an unknown function (e.g., with respect to clinical effect).

In some embodiments, a novel compound that satisfies the constraint receives a classification score from the classifier that is equal to or greater than the classification score for one or more compounds in the first plurality of compounds in the first training dataset.

In some embodiments, the classification of the respective candidate compound is obtained according to the updated first and second plurality of weights associated with the trained neural network encoder and the classifier, respectively.

In some embodiments, the method further comprises using a second classifier. In some such embodiments, the method comprises training and using a second classifier to obtain a classification for a second biological property other than the first biological property. In some such embodiments, the second biological property includes, but is not limited to, toxicity, off-target effects, solubility, molecular weight, and/or any combination thereof. In some such embodiments, the second classifier is applied before or after the decoding of the candidate projections.

In some embodiments, the second classifier is any of the classifiers disclosed in greater detail herein (see, “Constrained Representation Learning,” above). For example, in some embodiments, the second classifier is, for example, a logistic regression classifier, a k-nearest neighbor classifier, a deep neural network classifier, a support vector machine classifier, a decision tree classifier, or a naïve Bayes classifier, etc.

Referring to Block 216, in some embodiments, the using the trained neural network encoder, trained classifier, and trained decoder further comprises verifying a first compound in the plurality of candidate compounds has the first biological property by a third procedure that comprises subjecting the first compound to a wet lab assay that verifies that the respective candidate compound has the first biological property. For example, in some embodiments, the wet lab assay is a compound activity assay. For example, in some embodiments, the wet lab assay is an estrogen receptor alpha (ER-alpha) compound screening assay and/or an auto-fluorescence counter screen, where the auto-fluorescence counter screen is performed as a proxy for toxicity-dependent cell death. In some embodiments, the wet lab assay is an aryl hydrocarbon receptor (AhR) antagonist mode assay and/or a cell viability counter screen. In some embodiments, the wet lab assay is selected from the group consisting of an estrogen receptor alpha (ER-alpha) compound screening assay, an aryl hydrocarbon receptor (AhR) antagonist mode assay, an aromatase antagonist mode assay, an androgen receptor (AR) assay, peroxisome proliferator-activated receptor gamma (PPAR-gamma) agonist mode assay, a nuclear factor (erythroid-derived 2)-like 2/antioxidant responsive element (Nrf2/ARE) mode assay, a heat shock factor response element (HSE) mode assay, an ATAD5 mode assay, a mitochondrial membrane potential (MMP), a p53 mode assay, a cell viability counter screen, and/or an auto-fluorescence counter screen. Further assays are contemplated, as are described in Huang et al. 2016, “Modelling the Tox21 10 K chemical profiles for in vivo toxicity prediction and mechanism characterization,” Nat Commun. 7, p. 10425, which is hereby incorporated by reference.

In some embodiments, the wet lab assay is performed using one or more cell lines, including human cell lines, animal (e.g., hamster, chicken, rat, and/or mouse) cell lines, and/or one or more tissue types (e.g., liver, kidney, ovarian, cervical cancer, breast cancer, and/or colon cancer). In some embodiments, the biological property is measured in a healthy cell line and/or an unhealthy cell line (e.g., a cancerous cell line). In some embodiments, a cell line is selected from the group consisting of HepG2, ME-180, HEK293, MDA-MB-453, MCF-7, CHO, DT40, BG1, HeLa, GH3, HCT-116, C3H10T1/2, and NIH/3T3. In some embodiments, the wet lab assay is performed using at least 1, at least 2, at least 3, at least 4, at least 5, at least 6, at least 7, at least 8, at least 9, or at least 10 cell lines. In some embodiments, the wet lab assay comprises any assay known in the art, including, but not limited to, colorimetric, fluorescence, bioluminescence, and resonance energy transfer (FRET). In some embodiments, the wet lab assay comprises high-throughput screening (HTS) and/or high-content screening (HCS) methods.

In some embodiments, the wet lab assay comprises determining a change in cytotoxicity, cell viability, gene toxicity, developmental toxicity, and/or mitochondrial toxicity in response to an agonism and/or antagonism of one or more cellular-components of interest (e.g., AhR, AP-1, AR-BLA, ARE, AR-MDA, aromatase, CAR, caspases (e.g., caspase-3/7), ATAD5, ER-beta, ER-BLA, ER-BG1, ERR, ER stress, FXR-BLA, TR-beta, GR-BLA, H2AX, HDAC. HRE-BLA, HSE-BLA, NFkB, P53, PGC-ERR, PPAR-delta-BLA, PPAR-gamma, PR-BLA, PXR, RAR, ROR. RXR-BLA, SBE-BLA (TGF-beta), Hedgehog, TRHR, TSHR, and/or VDR-BLA). Other methods for measuring and/or verifying biological properties are contemplated, for example as described in Huang R, 2016, “A Quantitative High-Throughput Screening Data Analysis Pipeline for Activity Profiling,” High-Throughput Screening Assays in Toxicology, Methods in Molecular Biology; 1473(1); Huang et al., 2016, “Modelling the Tox21 10 K chemical profiles for in vivo toxicity prediction and mechanism characterization,” Nat Commun. 7, p. 10425; and Huang et al., 2018. “Expanding biological space coverage enhances the prediction of drug adverse effects in human using in vitro activity profiles,” Sci Rep. 8(1):3783, each of which is hereby incorporated herein by reference in its entirety, and/or any substitutions, additions, deletions, modifications, and/or combinations thereof as will be apparent to one skilled in the art.

Referring to Block 218, the verifying further comprises synthesizing the first compound.

In some embodiments, the method further comprises subjecting the respective candidate compound to a wet lab assay that verifies that the respective candidate compound has the first biological property. In some embodiments, the verifying further comprises synthesizing the respective candidate compound.

In some embodiments, the method comprises verifying that a first compound in the plurality of candidate compounds has one or more biological properties. In some embodiments, the method comprises verifying at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 biological properties for a first compound in the plurality of candidate compounds. In some embodiments, the method comprises verifying at least a first biological property for each compound in a plurality of candidate compounds, where the plurality of candidate compounds comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 candidate compounds. In some embodiments, the method comprises verifying at least a first biological property for each compound in a plurality of candidate compounds, where the plurality of candidate compounds comprises at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, or at least 10,000 candidate compounds.

Another aspect of the present disclosure provides a method of synthesizing a test compound that has a first biological property, where the test compound was designed by a method. The method comprises, at a computer system comprising at least one processor and a memory storing at least one program for execution by the at least one processor, at least one program comprising instructions for obtaining a first training dataset, in electronic form. The first training dataset comprises, for each respective compound in a first plurality of compounds (e.g., comprising 100 or more compounds), information regarding a chemical structure of the respective compound and one or more biological properties, in a plurality of biological properties, of the respective compound, and the plurality of biological properties includes the first biological property.

In this aspect, the method further comprises training an untrained or partially untrained neural network encoder and an untrained or partially untrained classifier by performing a first procedure. The first procedure comprises, for each respective compound in the first plurality of compounds, projecting the information regarding the chemical structure of the respective compound into a latent representation space in accordance with a first plurality of weights associated with the untrained or partially untrained neural network encoder to obtain a corresponding projected representation of the respective compound, and inputting the corresponding projected representation of the respective compound into the untrained or partially untrained classifier to obtain a classification of the respective compound in accordance with a second plurality of weights associated with the untrained or partially untrained classifier. The first procedure further comprises updating the first plurality of weights and the second plurality of weights by comparing the classification of each respective compound in the first plurality of compounds to the one or more biological properties of the respective compound in the first training dataset thereby obtaining a trained neural network encoder and a trained classifier.

The method further comprises obtaining a second training dataset, in electronic form, where the second training dataset comprises, for each respective compound in a second plurality of compounds (e.g., comprising 100 or more compounds), information regarding a chemical structure of the respective compound. The method further comprises training an untrained or partially untrained decoder by performing a second procedure that comprises, for each respective compound in the second plurality of compounds, projecting the information regarding the chemical structure of the respective compound into a latent representation space in accordance with the first plurality of weights associated with the trained neural network encoder to obtain a corresponding projected representation of the respective compound, and inputting the corresponding projected representation of the respective compound into the untrained or partially untrained decoder to obtain a chemical structure of the respective compound in accordance with a third plurality of weights associated with the untrained or partially untrained decoder. The second procedure further comprises updating the third plurality of weights by comparing the chemical structure of each respective compound outputted by the untrained or partially untrained decoder to the actual chemical structure of the respective compound from the second training dataset thereby obtaining a trained decoder.

The method further comprises using the trained neural network encoder, trained classifier, and trained decoder to identify a test compound that has the first biological property, wherein the test compound is not present in the first and second training set.

In some embodiments, the method of synthesizing a test compound that has a first biological property further comprises designing a test compound that has a first biological property using a trained neural network encoder, trained classifier, and trained decoder to identify a test compound that has a first biological property, where the trained neural network encoder, trained classifier, and trained decoder were trained by any of the methods and embodiments described herein and/or any combinations or alternatives thereof as will be apparent to one skilled in the art.

In some embodiments, the method of synthesizing a test compound that has a first biological property further comprises any of the methods or embodiments for discovering a test compound that has a first biological property described herein and/or any combinations or alternatives thereof as will be apparent to one skilled in the art.

Another aspect of the present disclosure provides a computer system for performing a method for discovering a test compound that has a first biological property. In this aspect, the computer system comprises one or more processors and memory, the memory storing instructions for performing a method for discovering a test compound that has a first biological property. In some embodiments, the memory stores instructions for performing any of the methods and embodiments described herein and/or any combinations or alternatives thereof as will be apparent to one skilled in the art.

Another aspect of the present disclosure provides a non-transitory computer-readable medium storing one or more computer programs, executable by a computer, for performing a method for discovering a test compound that has a first biological property. In this aspect, the computer comprises one or more processors and a memory, the one or more computer programs collectively encoding computer executable instructions that perform a method. In some embodiments, the computer executable instructions perform any of the methods and embodiments described herein and/or any combinations or alternatives thereof as will be apparent to one skilled in the art.

### Additional Embodiments

Compounds.

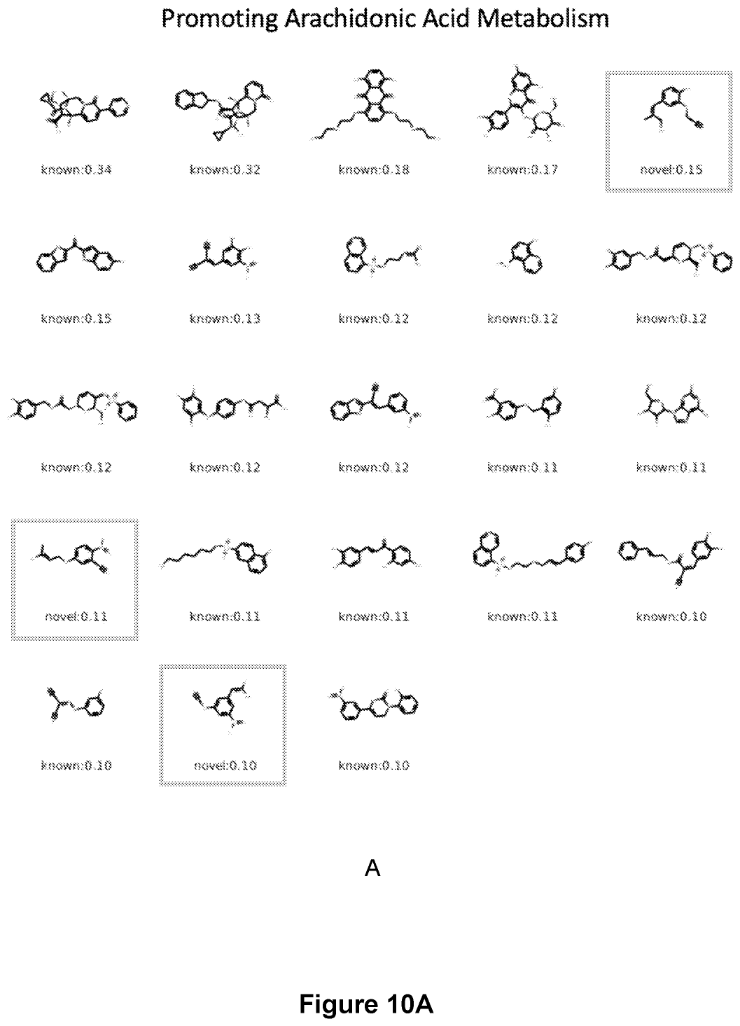

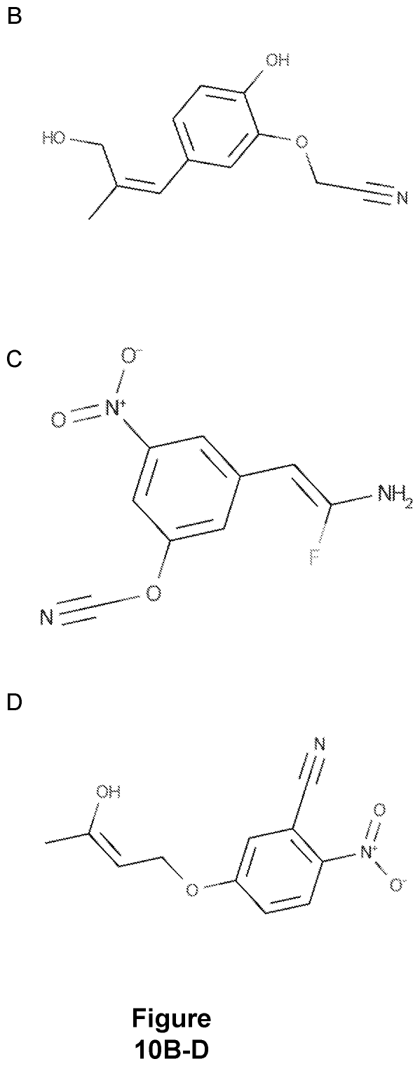

Another aspect of the present disclosure provides a compound selected from the compound structures provided in FIGS. 10A-D, and/or any derivatives or pharmaceutically acceptable salts thereof. In some embodiments, the compound is selected from the compounds depicted in FIGS. 10B, 10C, and/or 10D. In some embodiments, the compound has a compound structure represented by a SMILES string selected from the group consisting of C1=C(C═C(C═C1C═C(F)N)[N+](═O)[O−])OC #N; C1(=CC(═C(C═C1)[N+]([O−])═O)C #N)OCC═C(C)O, and/or C1(=CC(=CC=C1O)C═C(C)CO)OCC #N. In some embodiments, the compound has a first biological property. In some embodiments, the first biological property is activation of arachidonic acid metabolism. In some embodiments, the compound is obtained using any of the methods and/or embodiments disclosed herein, and/or by any substitutions, additions, deletions, modifications, and/or combinations thereof as will be apparent to one skilled in the art. In some embodiments, the compound is used to modulate arachidonic acid metabolism in a cell.

Pharmaceutical Compositions.

Another aspect of the present disclosure provides a pharmaceutical composition comprising a compound selected from the compound structures provided in FIGS. 10A-D, and/or any derivatives or pharmaceutically acceptable salts thereof. In some embodiments, the compound has a first biological property. In some embodiments, the first biological property is activation of arachidonic acid metabolism. In some embodiments, the pharmaceutical composition comprises 1, 2, 3, 4, 5, 6, 7, 8, 9, 10, 11, 12, 13, 14, 15, 16, 17, 18, 19, 20, 21, 22, 23 or more than 23 compounds selected from the compound structures provided in FIGS. 10A-D, or any derivatives or pharmaceutically acceptable salts thereof.

In some embodiments, the pharmaceutical composition comprises a compound according to any one of the compounds described herein (see above; “Compounds”), or a pharmaceutically acceptable salt thereof, and a pharmaceutically acceptable carrier or diluent. In some embodiments, the pharmaceutical composition is a therapeutic composition for the treatment of a disorder. In some embodiments, the pharmaceutical composition is a therapeutic composition for the treatment of an inflammatory disorder.

In some embodiments, the pharmaceutical composition is formulated in accordance with standard pharmaceutical practice for use in a therapeutic combination for therapeutic treatment (including prophylactic treatment) of disorders (e.g., inflammatory disorders) in mammals including humans.

In some embodiments, the pharmaceutical composition encompasses a bulk composition and/or individual dosage units comprised of one or more pharmaceutically active agents (e.g., compounds as provided in FIGS. 10A-D), along with any pharmaceutically inactive excipients, diluents, carriers, or glidants. In some embodiments, the bulk composition and each individual dosage unit contain fixed amounts of the respective one or more pharmaceutically active agents. As used herein, a bulk composition refers to material that has not yet been formed into individual dosage units. For example, an illustrative dosage unit is an oral dosage unit such as tablets, pills, capsules, and the like. Similarly, in some embodiments, a method of treating a patient by administering a pharmaceutical composition includes the administration of the bulk composition and/or individual dosage units.

Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (generally recognized as safe; GRAS) to be administered to a mammal (e.g., a human). In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of a pharmaceutically active agent (e.g., any one or more of a compound as described herein, any one or more of the compounds provided in FIGS. 10A-D, and/or any combination of the same) or aid in the manufacturing of a pharmaceutical product (e.g., medicament).

In some embodiments, the pharmaceutical composition includes formulations comprising a carrier suitable for the desired delivery method. Suitable carriers include any material that when combined with the pharmaceutical composition retains the anti-tumor function of the pharmaceutical composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16th Edition, A. Osal., Ed., 1980). In some embodiments, the pharmaceutical composition includes formulations suitable for a specific administration route (e.g., any one or more of the methods of administration provided herein). Techniques and formulations are known in the art (see, Remington's Pharmaceutical Sciences 18th Edition, Mack Publishing Co., Easton, Pa., 1995).

For example, a formulation for a pharmaceutical composition suitable for oral administration can be prepared as discrete units such as pills, hard or soft, e.g., gelatin capsules, cachets, troches, lozenges, aqueous or oil suspensions, dispersible powders or granules, emulsions, syrups or elixirs, each containing a predetermined amount of a compound and/or a conjugate disclosed herein. In some embodiments, such formulations are prepared according to any method known to the art for the manufacture of pharmaceutical compositions, where such compositions contain one or more agents including sweetening agents, flavoring agents, coloring agents and preserving agents, in order to provide a palatable preparation. In some embodiments, compressed tablets are prepared by compressing in a suitable machine a pharmaceutically active agent (e.g., any one or more of a compound as described herein, any one or more of the compounds provided in FIGS. 10A-D, and/or any combination of the same) in a free-flowing form such as a powder or granules, optionally mixed with a binder, lubricant, inert diluent, preservative, surface active or dispersing agent. In some embodiments, molded tablets are made by molding in a suitable machine a mixture of the powdered drug and/or pharmaceutically active agent moistened with an inert liquid diluent. The tablets can optionally be coated or scored and optionally are formulated so as to provide slow or controlled release of the drug and/or pharmaceutically active agent therefrom.

In some embodiments, a formulation for a pharmaceutical composition suitable for treatment of the eye or other external tissues (e.g., mouth and skin) can be applied as a topical ointment or cream containing the pharmaceutically active agent (e.g., any one or more of a compound as described herein, any one or more of the compounds provided in FIGS. 10A-D, and/or any combination of the same). In some embodiments, the formulation is an ointment, where the pharmaceutically active agent is employed with either a paraffinic or a water-miscible ointment base. Alternatively, in some embodiments, the pharmaceutically active agent is formulated in a cream with an oil-in-water cream base.

In some embodiments, a formulation for a pharmaceutical composition is an aqueous suspension comprising the pharmaceutically active agent (e.g., any one or more of a compound as described herein, any one or more of the compounds provided in FIGS. 10A-D, and/or any combination of the same) and excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). In some embodiments, the aqueous suspension further comprises one or more preservatives such as ethyl or n-propyl p-hydroxybenzoate, one or more coloring agents, one or more flavoring agents, and/or one or more sweetening agents, such as sucrose or saccharin.

In some embodiments, the pharmaceutical composition is in the form of a sterile injectable preparation, such as a sterile injectable aqueous or oleaginous suspension. In some embodiments, the suspension is formulated according to the known art using suitable dispersing or wetting agents and suspending agents as described above. In some embodiments, the sterile injectable preparation is a solution or a suspension in a non-toxic parenterally acceptable diluent or solvent, such as a solution in 1,3-butanediol or prepared from a lyophilized powder. Suitable vehicles and solvents include water, Ringer's solution and isotonic sodium chloride solution. In addition, the sterile injectable preparation can comprise sterile fixed oils as a solvent or suspending medium, any bland fixed oil including synthetic mono- or diglycerides, and/or fatty acids such as oleic acid.

Additional embodiments of pharmaceutical compositions are possible, including any additions, deletions, substitutions, and/or modifications of the foregoing examples, as will be apparent to one skilled in the art, and/or any combinations thereof.

Modulation of Arachidonic Acid Metabolism.

Another aspect of the present disclosure provides a method of modulating arachidonic acid metabolism in a cell, comprising contacting the cell with a compound according to any one of the compounds disclosed herein and/or provided in FIGS. 10A-D (see the above section: “Compounds”), or a pharmaceutically acceptable salt thereof.

In some embodiments, the cell is a mammalian cell.

In some embodiments, the cell is a human cell.

In some embodiments, the modulating arachidonic acid metabolism comprises activation of the arachidonic acid metabolism pathway. In some embodiments, the modulating arachidonic acid metabolism comprises an activation or a repression of one or more intermediates in the arachidonic acid metabolism pathway. In some embodiments, the modulating arachidonic acid metabolism comprises a change in expression level of one or more intermediates in the arachidonic acid metabolism pathway. Intermediates of the arachidonic acid metabolism pathway include, for example, any precursors, downstream products, and/or catalyzing enzymes including but not limited to arachidonic acid (AA), linoleic acid, gamma-linoleic acid, dihomo-gamma-linoleic acid, phospholipase A2 (PLA2), phospholipase C (PLC), phospholipase D (PLD), diacylglycerol (DAG), phosphatidylcholine, phosphatic acid, eicosanoids, isoprostanes, and/or phosphatidate phosphohydrolase. In some embodiments, the modulating arachidonic acid metabolic comprises a modulation of one or more enzymes and/or downstream products of arachidonic acid metabolism (e.g., via the cyclooxygenase, lipoxygenase, cytochrome p450 (CYP 450) and/or anandamide pathways). For example, in some embodiments, the one or more enzymes and/or downstream products involved in the cyclooxygenase pathway include COX-1, COX-2 (prostaglandin H synthase), prostaglandins (e.g., PGH2, PGE2, PGD2, PGF2alpha, and/or prostacyclins (e.g., PGI2), and/or thromboxanes (e.g., TXA2, TXB2). In some embodiments, the one or more enzymes and/or downstream products involved in the lipoxygenase pathway include LOX-5, LOX-8, LOX-12, LOX-15 enzymes and/or their products, leukotrienes (e.g., LTA4, LTB4, LTC4, LTD4 and/or LTE4), lipoxins (e.g., LXA4 and/or LXB4) and/or 8-12-15-hydroperoxyeicosatetraenoic acid (HPETE). In some embodiments, the one or more enzymes and/or downstream products involved in the CYP 450 pathway include CYP450 epoxygenase, CYP450 ω-hydroxylase, epoxyeicosatrienoic acid (EETs) and/or 20-hydroxyeicosatetraenoic acid (20-HETE). In some embodiments, the one or more enzymes and/or downstream products involved in the anandamide pathway comprises FAAH (fatty acid amide hydrolase), endocannabinoid, and/or anandamide. See, for example, Hanna and Hafez, 2018, “Synopsis of arachidonic acid metabolism: A review,” J Adv Res 11:23-32: doi: 10.1016/j.jare.2018.03.005, which is hereby incorporated herein by reference in its entirety.

Therapeutic Applications.

Another aspect of the present disclosure further provides a method of stimulating an immune response in a subject in need thereof comprising administering to the subject an effective amount of a compound according to any one of the compounds disclosed herein and/or provided in FIGS. 10A-D (see the above section: “Compounds”), or a pharmaceutically acceptable salt thereof. Arachidonic acid, for example, has been reported to play a major role in the maintenance of the immune system, including allergies and inflammation, as well as in the resolution of inflammatory processes. See, for example, Hanna and Hafez, 2018, “Synopsis of arachidonic acid metabolism: A review,” J Adv Res 11:23-32; doi: 10.1016/j.jare.2018.03.005, which is hereby incorporated herein by reference in its entirety.

In some embodiments, the present disclosure further provides a method of stimulating an immune response in a subject in need thereof comprising administering to the subject a pharmaceutical composition comprising an effective amount of a compound according to any one of the compounds disclosed herein and/or provided in FIGS. 10A-D (see the above section; “Compounds”), or a pharmaceutically acceptable salt thereof.

In some embodiments, the administering modulates the arachidonic acid metabolism pathway in a cell. In some embodiments, the stimulating the immune response comprises modulating the arachidonic acid metabolism pathway in a cell. In some embodiments, the stimulating the immune response comprises contacting a cell with a compound and/or pharmaceutical composition as disclosed herein.

In some embodiments, the subject is a mammal. In some embodiments, the subject is a human (e.g., a human with an arachidonic acid metabolism disorder).

Another aspect of the present disclosure further provides a method of treating a disorder (e.g., an arachidonic acid deficiency, an arachidonic acid metabolism disorder, and/or an inflammatory disorder) in a subject in need thereof comprising administering to the subject an effective amount of a compound according to any one of the compounds disclosed herein and/or provided in FIGS. 10A-D (see the above section; “Compounds”), or a pharmaceutically acceptable salt thereof.

In some embodiments, the present disclosure further provides a method of treating a disorder (e.g., an arachidonic acid deficiency, an arachidonic acid metabolism disorder, and/or an inflammatory disorder) in a subject in need thereof comprising administering to the subject a pharmaceutical composition comprising an effective amount of a compound according to any one of the compounds disclosed herein and/or provided in FIGS. 10A-D (see the above section; “Compounds”), or a pharmaceutically acceptable salt thereof.

In some embodiments, the administering modulates the arachidonic acid metabolism pathway in a cell. In some embodiments, the treating the disorder comprises modulating the arachidonic acid metabolism pathway in a cell. In some embodiments, the treating the disorder comprises contacting a cell with a compound and/or pharmaceutical composition as disclosed herein.

In some embodiments, the subject is a human. In some embodiments, the subject is a human that has been diagnosed with a disorder (e.g., an arachidonic acid deficiency, an arachidonic acid metabolism disorder, and/or an inflammatory disorder).

In some embodiments, an effective amount of a compound and/or a pharmaceutical composition comprising the same, is administered to the subject by any suitable means to modulate the respective pathway, stimulate the immune response and/or treat the respective disorder. For example, in certain embodiments, the compound and/or pharmaceutical composition can be administered by intravenous, intraocular, subcutaneous, and/or intramuscular means. The compound and/or pharmaceutical composition can be administered by parenteral (including intravenous, intradermal, intraperitoneal, intramuscular and subcutaneous) routes or by other delivery routes, including oral, nasal, buccal, sublingual, intra-tracheal, transdermal, transmucosal, and pulmonary. In certain embodiments, the compound and/or pharmaceutical composition can be administered either systemically or locally (e.g., directly). Systemic administration includes: oral, transdermal, subdermal, intraperitioneal, subcutaneous, transnasal, sublingual, or rectal. Alternatively, the compound and/or pharmaceutical composition can be delivered via a sustained delivery device implanted, for example, subcutaneously or intramuscularly. The compound and/or pharmaceutical composition can be administered by continuous release or delivery, using, for example, an infusion pump, continuous infusion, controlled release formulations utilizing polymer, oil or water insoluble matrices.

In certain embodiments, the term “effective amount” refers to an amount of a compound and/or pharmaceutical composition that results in a desired biological or physiological effect (e.g., modulation of the arachidonic acid metabolism pathway and/or stimulation of an immune response) and/or improvement or remediation of disease or condition in the subject (e.g., an arachidonic acid deficiency). An effective amount to be administered to the subject can be determined by a physician with consideration of individual differences in age, weight, the disease or condition being treated, disease severity and response to the therapy. In certain embodiments, the compound and/or pharmaceutical composition can be administered to a subject alone or in combination with other compositions. In some embodiments, the compound and/or pharmaceutical composition is administered at periodic intervals, over multiple time points, and/or for a duration of treatment. For example, in some such embodiments, the compound and/or pharmaceutical composition is administered at least every 1, 2, 3, 4, 6, 8, 12, or 24 hours, at least every 1, 2, 3, 4, 5, 6, or 7 days, at least every 1, 2, 3 or 4 weeks, or at least at a monthly, bi-monthly, annually or bi-annually frequency. In some embodiments, the compound and/or pharmaceutical composition is administered at a single time point. In some embodiments, the time needed to complete a course of the treatment is determined by a physician. In some embodiments, the course of treatment ranges from as short as one day to more than a month. In certain embodiments, a course of treatment can be from 1 to 6 months, or more than 6 months.

In some embodiments, the compound and/or pharmaceutical composition comprises a formulation that is selected for the mode of delivery, e.g., intravenous, intraocular, subcutaneous, and/or intramuscular means.

According to some embodiments of the present invention, the compound and/or pharmaceutical composition can be administered in combination with one or more active therapeutic agents for treating co-infections or associated complications. Additional methods of administration of compounds and/or pharmaceutical compositions are possible, as will be apparent to one skilled in the art.

### III. EXAMPLES

### Example 1. Predicting Molecules that Effect Cell State Transitions

The following describes a sequence of proof of concepts that illustrate the abovementioned systems and methods and provide first demonstrations of predicted in silico-generated molecules with desired effects on pathways and cell states.

From Predicting Known Drugs to Generating New Molecules.

International Patent Application No. PCT/US2019/041976, entitled “Methods of Analyzing Cells,” filed Jul. 16, 2019, which is hereby incorporated herein by reference in its entirety, discloses the prediction of known drugs that effect desired cell state changes by learning mappings from molecular datasets that capture disease-relevant cell states (e.g., cellular phenotypes) to molecular datasets that capture perturbation experiments of known molecules. Using such methods, molecules that would be most likely to induce or revert a desired disease-relevant molecular state can be inferred by predicting the respective identities in the form of labels for the respective molecules out of a perturbation dataset comprising tens of thousands of molecules.

The procedure is generalized by encoding drug labels into representations of their chemical structure, which allows the interpolation of drugs between desired states and other constraints by varying their chemical structure.

Labelling Chemical Structures with Cellular State Activations or Inhibitions.

One approach of doing so is to “collapse” all information in the molecular feature space (e.g., the transcriptional profile as measured in scRNA-seq or the L1000 assay), to a single “score” that represents the activation of a cell state. Specifically, classifiers are trained on data with disease relevance on the task of discriminating relevant cell states. Alternatively, differential expression tests are performed to derive gene sets marking the activation of a cell state. Applying such classifiers to a dataset capturing perturbation experiments of molecules amounts to labelling drugs with a score that indicates whether the drug activated or inhibited the cell state, depending on covariates. Alternatively, if using differential-expression derived gene sets, the score can be computed using, e.g., Scanpy.

Through this procedure, all high-dimensional variation in the molecular configuration is eliminated, resulting in a table that stores molecules (e.g., drugs) in the first column and activations and inhibitions of cell states and/or additional covariates in subsequent columns. The column containing the drug (molecule) label can be readily replaced with its chemical structure, for instance, in the form of its SMILES string. In some preferred embodiments, the representation of chemical structures as SMILES strings facilitates the application of the methods for learning mappings of chemical structure to cell state labels (see, International Patent Application No. PCT/US2019/041976, entitled “Methods of Analyzing Cells,” filed Jul. 16, 2019, which is hereby incorporated herein by reference in its entirety).

Interpolation of Compounds (Known Drugs).

New molecule generation needs new latent space vectors. Provided with these mappings, existing data between cell states is interpolated to generate new molecules. While many approaches for such interpolations exist, they are all followed by a quality assessment of the produced molecule using the classifier described previously. Even in the presence of a suboptimal interpolation schema (“generator”), the classifier can ensure that one in fact only keeps molecules that induce the desired cell state change.

The interpolation can be performed by variety of ways, such as sampling a pair of known molecules that have the desired activity and taking steps on the line that connects their latent space vector representations. Alternatively, in some embodiments, a Generative Adversarial Network (GAN) is used to learn a mapping from high-dimensional Gaussian noise to the latent vector space, such that, when added to the representation of known active molecules, the newly obtained latent vector still generates an active molecule. In another embodiment, the interpolation is performed for a plurality of P molecules (e.g., where P is greater than 2). In some such embodiments, where P is greater than 2, the interpolation is performed by determining the center of mass for the plurality of P molecules, selecting a molecule from the plurality of P molecules (e.g., via random sampling), and applying a linear interpolation described above to the pair represented by the randomly selected molecule and the center of mass for the plurality of P molecules. In some embodiments, the random sampling followed by linear interpolation method is repeated M times to generate a plurality of molecules (e.g., M generated molecules). In some embodiments, P is an integer with a value of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 10. In some embodiments, P is an integer with a value of at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 800, at least 9000, or at least 10,000. In some embodiments, M is an integer less than or equal to P. In some embodiments, M is an integer greater than or equal to P.

Predicting Drugs that Activate States Characterized by the Activation of Given Pathways.

Quantifying the activation of a pathway in a cellular state is often an important characterization of a state with high disease relevance. The following considered this specific case of representing cellular states, based on known pathways that are characterized by known gene sets. The procedure can be equally well executed with gene sets derived de novo from molecular data with disease relevance.

The gene sets that were used to define 236 pathways were obtained from the KEGG database. The perturbation experiment data were obtained from LINCS L1000 assay (Level 5) for A549 cells. The selected data were further filtered to include only those where the perturbation was applied for 24 hours, resulting in 16377 perturbations from 10600 small molecules. The replicates for the same small molecule were averaged. A random 600 out of 10600 small molecules were held out from training, creating a test dataset. The remaining data was referred to as the training dataset.

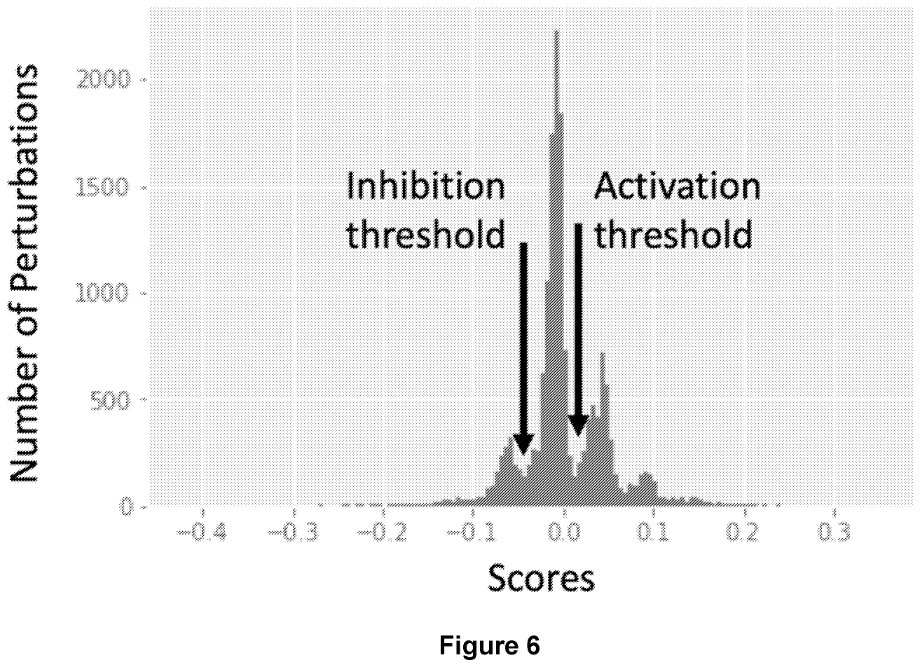

Each perturbation experiment was scored for each pathway by providing the associated gene sets to a Python package, Scanpy. After scoring each perturbation for each pathway, binary labels was created such that if the score for a particular pathway was negative and less than the inhibition threshold, the small molecule was considered to inhibit that pathway. Likewise, if the score was higher than the activation threshold, it was considered to promote the pathway of interest. The inhibition and activation thresholds were defined from the multimodality of the score distribution across the perturbations for a given pathway. For example, FIG. 6 illustrates the score distribution for inhibition and activation thresholds for perturbations applied to the mTOR pathway. Scoring was performed using K-means clustering algorithm with 3 clusters on the scores of each pathway, resulting in low, medium, and high score clusters which define the inhibition and activation thresholds. Therefore, each perturbation experiment is labeled with 236 activation and 236 inhibition binary labels.

Small molecules were initially represented as SMILES strings. These SMILES strings were converted into molecular graph representation using a common Python library, RDKIT (see, for example, RDkit: Open-source cheminformatics; available on the Internet at www.rdkit.org). A molecular graph is a data structure which contains an adjacency matrix and a feature matrix. The adjacency matrix is a symmetric binary matrix where rows (and columns) correspond to the atoms in the molecule and entries of the matrix indicate if there is a bond between the pair of atoms corresponding row and column. In contrast, the feature matrix comprises the same number of rows, where each row represents the features of the corresponding atoms and the columns represent individual features across the atoms.

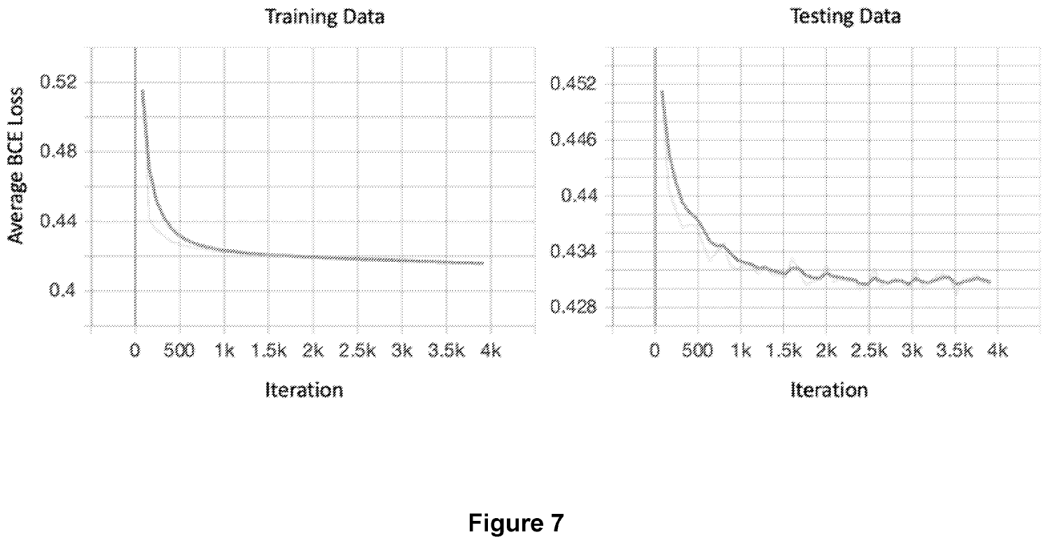

Molecules were encoded in 50-dimensional space via processing through the Graph Neural Network encoder model. A classifier was applied to predict the 472 binary labels of 236 pathways. The encoder and the classifier models were jointly trained to minimize the average binary cross entropy loss. For example, FIG. 7 illustrates loss curves over multiple iterations during training. In some embodiments, overfitting of a model to a training dataset (e.g., the training samples) is observed by an increase in test data loss and/or a decrease in test data accuracy. Such overfitting indicates a loss in the ability of the model to generalize in order to generate predictions from the test dataset. Thus, in some embodiments, training a model comprises monitoring loss or accuracy curves over one or more periods of training time (e.g., epochs) to assess whether overfitting of the model has occurred. In Example 7, the model converges without losing generalization ability to unseen (testing) data (e.g., overfitting).

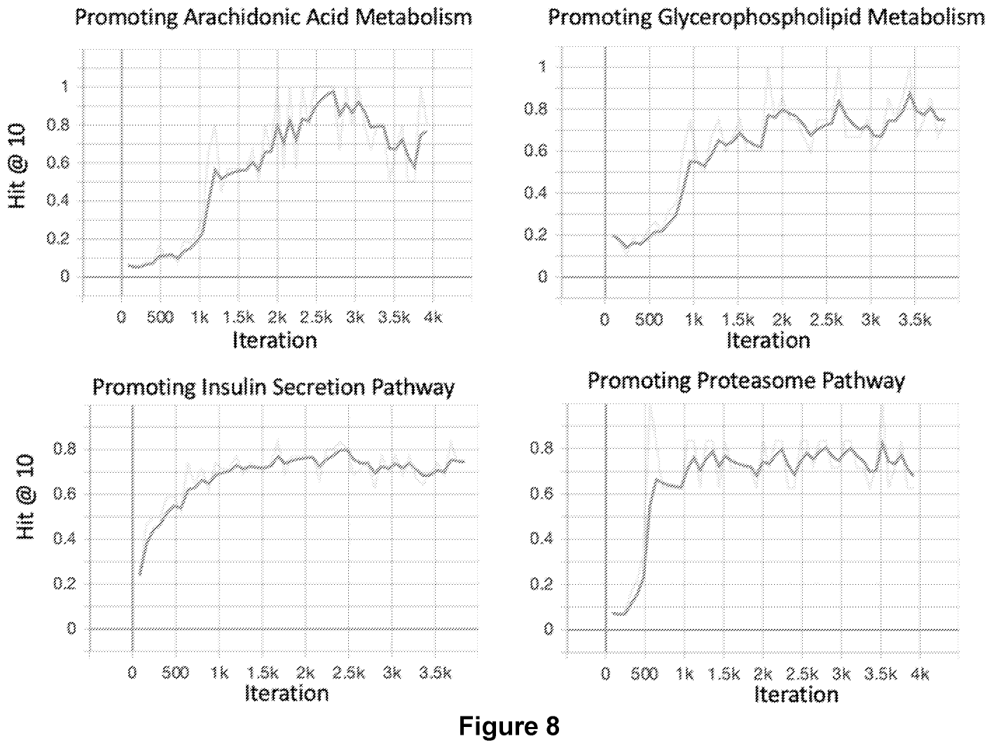

After training the model, the performance was measured via the precision at 10% recall score, where precision indicates the relevance of the returned results (e.g., specificity) and recall indicates the number of relevant results returned (e.g., sensitivity). Out of 472 cases, the model achieved reliably high precision (>80%) for 12 pathways. FIG. 8 illustrates the performance of the model using four example pathways, showing relatively high precision (80% or higher) at 10% recall.

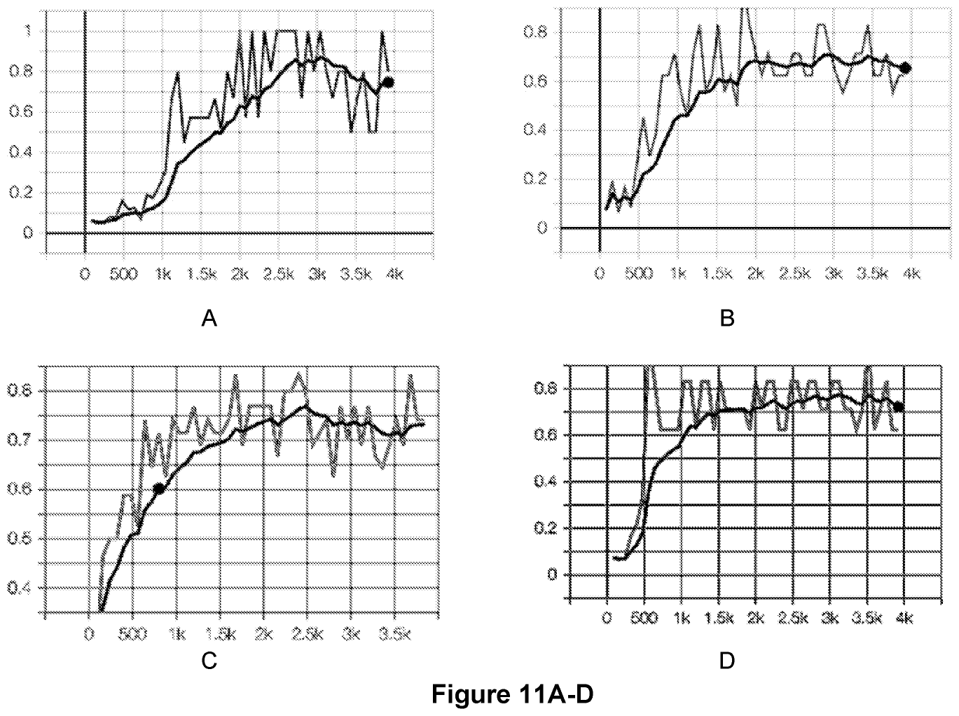

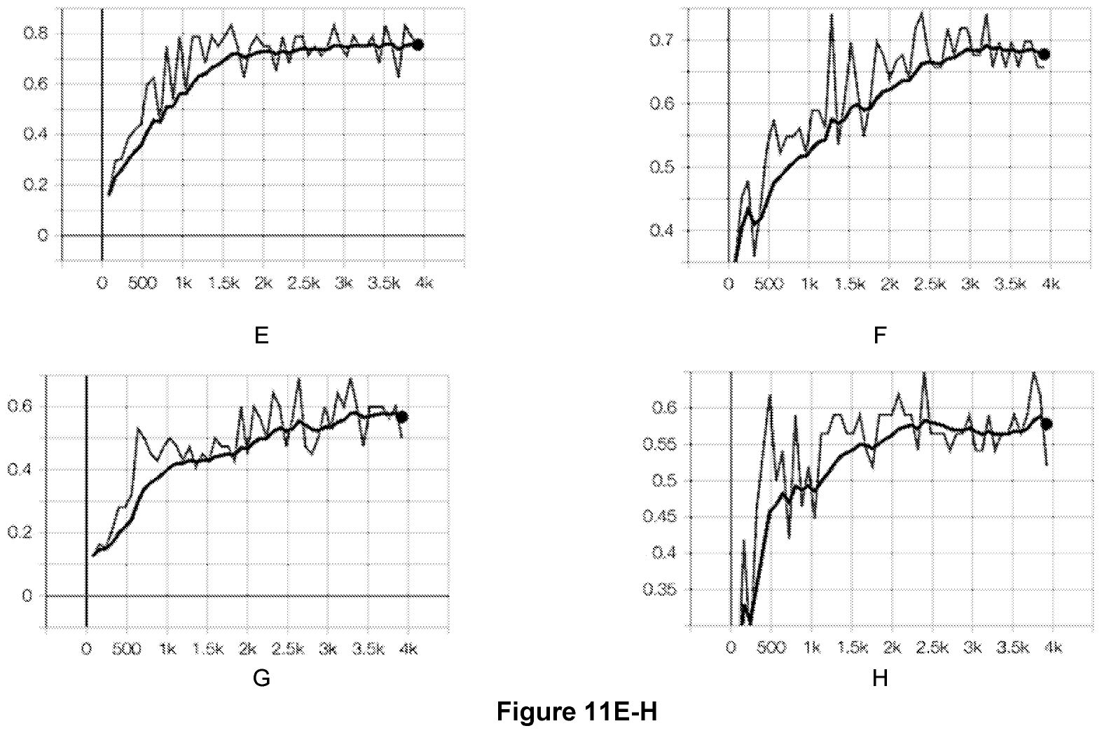

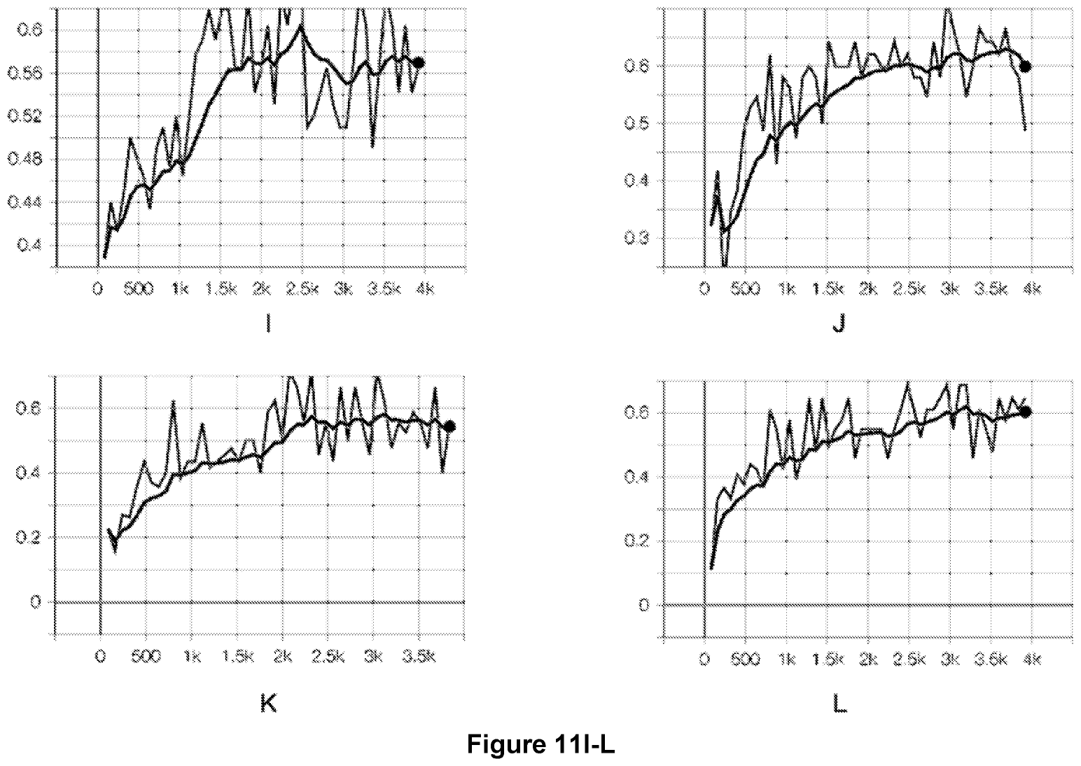

More specifically. FIGS. 11A-L illustrate performance of the classification model using each of the 12 pathways. FIG. 11A: activation of arachidonic acid metabolism; 11B: inhibition of alpha-linolenic acid metabolism; 11C: activation of insulin secretion; 11D: activation of proteasome; 11E: activation of synaptic vesicle cycle; 11F: inhibition of human T-cell leukemia virus 1 infection; 11G: activation of cytosolic DNA sensing pathway; 11H: inhibition of calcium signaling pathway; 11I: inhibition of Chagas disease (e.g., American trypanosomiasis); 11J: inhibition of oocyte meisosis; 11K: inhibition of nucleotide excision repair; 11L: activation of pancreatic secretion. For each of the 12 pathways, the model exhibited high precision (e.g., 60°/o or higher) at 10% recall, where precision improved over higher numbers of training iterations.

Molecule generation was performed using a decoder that accepts the encoded molecule and corresponding junction tree representation as input and returns the corresponding SMILES string as output. The decoder (e.g., the generator) was trained on a ZINC dataset, which contains ˜250K drug like virtual molecules. See, for example, Irwin and Shoichet, “ZINC—A Free Database of Commercially Available Compounds for Virtual Screening,” J Chem Inf Model. 2005; 45(1): 177-182, which is hereby incorporated herein by reference in its entirety. To align the latent representation space of encoder and decoder, training the decoder was based on the pre-trained, parameter-frozen encoder with an objective to maximize the likelihood of molecular subgraph generation.

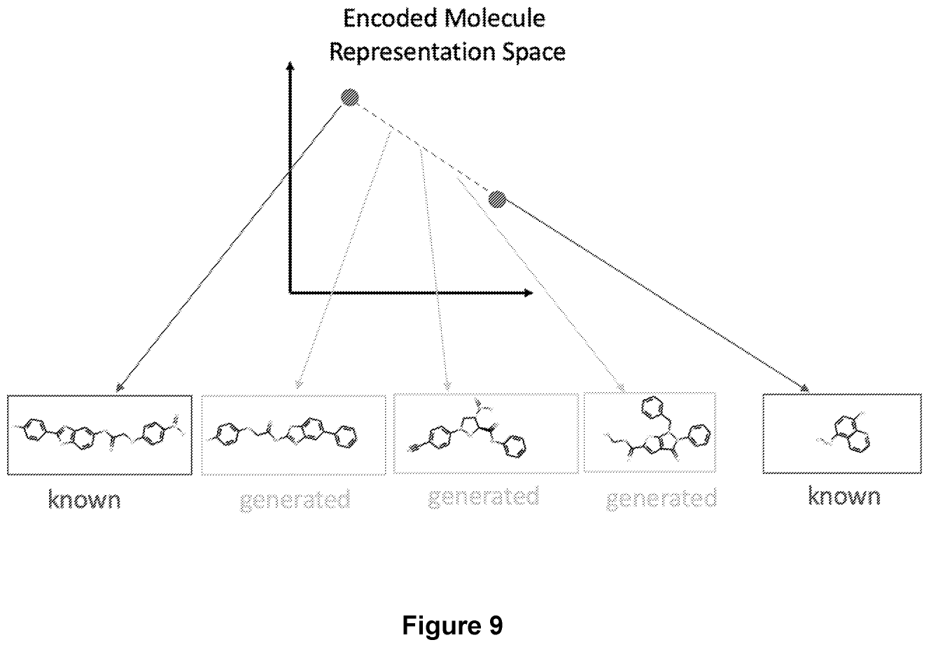

After training the decoder, the molecules that are known to promote particular pathways (e.g., promoting Arachidonic acid metabolism) were selected (e.g., the inference set) and encoded into the latent representation space. A pair of molecule representation vectors from this space was selected and new vectors were generated in this space along the line connecting the two molecules by interpolating with a desired amount. In some embodiments, interpolation between a pair of molecules and/or representation of molecules comprises selecting a number of desired intermediates (e.g., “steps”), along the line that connects the pair, and, at each respective “step,” predicting (e.g., generating) a new molecule and/or representation of a molecule. In some embodiments, the number of desired intermediates is at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10,000, or more than 10,000 intermediates. In an example embodiment, new vectors were generated in the space between the pair of molecule representation vectors by generating a molecule for each of 1000 intermediate points along the connecting line (e.g., “steps”). For example, FIG. 9 illustrates an example of molecules generated using interpolation of two molecule representation vectors corresponding to two known compounds with activity in promoting Arachidonic acid metabolism. These interpolated vectors were passed to the decoder to generate new SMILES strings, which were filtered to remove those that corresponded to known molecules. The remaining molecules were passed through the encoder and subsequently to the previously trained classifier in order to score their potential activation for the respective particular pathway (e.g., promoting Arachidonic acid metabolism). The molecules were thus further filtered by applying a retainment score threshold defined by precision at 10% recall (hits@10). FIG. 10A illustrates a set of molecules that promote Arachidonic acid metabolism, sorted by their scores from the classifier. In addition to known molecules, three novel molecules that were not present in either the training set or the inference set were generated by the model (shown in boxes and in FIGS. 10B, 10C, and 10D). Classifier scores were calculated as described above and as illustrated in FIG. 6.

Thus, as illustrated in FIGS. 8 and 11 A-L, in some embodiments, the present disclosure provides a classifier model for predicting (e.g., generating) one or more compounds with desired biological properties. In some embodiments, the model is trained using a plurality of molecules comprising at least the respective biological property (e.g., using cellular perturbation data obtained from a compound screening dataset). In some embodiments, the desired biological property is an ability to induce a perturbation in a cellular and/or biological pathway. For example, in some embodiments, the cellular and/or biological pathway is a pathway involved in arachidonic acid metabolism, alpha-linolenic acid metabolism, insulin secretion, proteasome, synaptic vesicle cycle, human T-cell leukemia virus 1 infection, cytosolic DNA sensing pathway, calcium signaling pathway, Chagas disease (e.g., American trypanosomiasis), oocyte meisosis, nucleotide excision repair, and/or pancreatic secretion. In some embodiments, the cellular and/or biological pathway is a pathway selected from the KEGG pathway database, available on the Internet at www.genome.jp/kegg/pathway.html. In some embodiments, the perturbation in the respective cellular and/or biological pathway is an activation and/or an inhibition in the respective pathway. Thus, in some embodiments, the present disclosure provides a classifier model for predicting (e.g., generating) compounds capable of activating and/or inhibiting a respective cellular and/or biological pathway, such as a pathway selected from the KEGG pathway database.

In some embodiments, the classifier model predicts (e.g., generates) at least 1, at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 compounds. In some embodiments, the classifier model predicts (e.g., generates) at least 50, at least 100, at least 200, at least 300, at least 400, at least 500, at least 600, at least 700, at least 800, at least 900, or at least 1000 compounds. In some embodiments, the classifier model predicts (e.g., generates) at least 1000, at least 2000, at least 3000, at least 4000, at least 5000, at least 10,000, or more than 10,000 compounds.

In some embodiments, the one or more compounds predicted by the classifier model comprises at least 1, at least 2, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, at least 500, at least 600, at least 700, at least 800, at least 900, or at least 1000 previously known compounds. In some embodiments, the one or more compounds predicted by the classifier model comprises no more than 1, no more than 2, no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, no more than 100, no more than 200, no more than 300, no more than 400, no more than 500, no more than 600, no more than 700, no more than 800, no more than 900, or no more than 1000 previously known compounds.

In some embodiments, the present disclosure provides a classifier model for predicting (e.g., generating) one or more compounds active in (e.g., induces a perturbation in) the arachidonic acid metabolism pathway. In some embodiments, the present disclosure provides a classifier model for predicting (e.g., generating) compounds that activate and/or inhibit the arachidonic acid metabolism pathway. In some embodiments, the present disclosure provides a method for (i) predicting (e.g., generating) one or more compounds that induce a perturbation in the arachidonic acid metabolism pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the present disclosure provides a classifier model for predicting (e.g., generating) one or more compounds active in (e.g., induces a perturbation in) the alpha-linolenic acid metabolism pathway. In some embodiments, the present disclosure provides a classifier model for predicting (e.g., generating) compounds that activate and/or inhibit the alpha-linolenic acid metabolism pathway. In some embodiments, the present disclosure provides a method for (i) predicting (e.g., generating) one or more compounds that induce a perturbation in the alpha-linolenic acid metabolism pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the present disclosure provides a classifier model for predicting (e.g., generating) one or more compounds active in (e.g., induces a perturbation in) the insulin secretion pathway. In some embodiments, the present disclosure provides a classifier model for predicting (e.g., generating) compounds that activate and/or inhibit the insulin secretion pathway. In some embodiments, the present disclosure provides a method for (i) predicting (e.g., generating) one or more compounds that induce a perturbation in the insulin secretion pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the present disclosure provides a classifier model for predicting (e.g., generating) one or more compounds active in (e.g., induces a perturbation in) the proteasome pathway. In some embodiments, the present disclosure provides a classifier model for predicting (e.g., generating) compounds that activate and/or inhibit the proteasome pathway. In some embodiments, the present disclosure provides a method for (i) predicting (e.g., generating) one or more compounds that induce a perturbation in the proteasome pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the present disclosure provides a classifier model for predicting (e.g., generating) one or more compounds active in (e.g., induces a perturbation in) the synaptic vesicle cycle pathway. In some embodiments, the present disclosure provides a classifier model for predicting (e.g., generating) compounds that activate and/or inhibit the synaptic vesicle cycle pathway. In some embodiments, the present disclosure provides a method for (i) predicting (e.g., generating) one or more compounds that induce a perturbation in the synaptic vesicle cycle pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the present disclosure provides a classifier model for predicting (e.g., generating) one or more compounds active in (e.g., induces a perturbation in) the human T-cell leukemia virus 1 infection pathway. In some embodiments, the present disclosure provides a classifier model for predicting (e.g., generating) compounds that activate and/or inhibit the human T-cell leukemia virus 1 infection pathway. In some embodiments, the present disclosure provides a method for (i) predicting (e.g., generating) one or more compounds that induce a perturbation in the human T-cell leukemia virus 1 infection pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the present disclosure provides a classifier model for predicting (e.g., generating) one or more compounds active in (e.g., induces a perturbation in) the cytosolic DNA sensing pathway. In some embodiments, the present disclosure provides a classifier model for predicting (e.g., generating) compounds that activate and/or inhibit the cytosolic DNA sensing pathway. In some embodiments, the present disclosure provides a method for (i) predicting (e.g., generating) one or more compounds that induce a perturbation in the cytosolic DNA sensing pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the present disclosure provides a classifier model for predicting (e.g., generating) one or more compounds active in (e.g., induces a perturbation in) the calcium signaling pathway. In some embodiments, the present disclosure provides a classifier model for predicting (e.g., generating) compounds that activate and/or inhibit the calcium signaling pathway. In some embodiments, the present disclosure provides a method for (i) predicting (e.g., generating) one or more compounds that induce a perturbation in the calcium signaling pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the present disclosure provides a classifier model for predicting (e.g., generating) one or more compounds active in (e.g., induces a perturbation in) the Chagas disease (e.g., American trypanosomiasis) pathway. In some embodiments, the present disclosure provides a classifier model for predicting (e.g., generating) compounds that activate and/or inhibit the Chagas disease (e.g., American trypanosomiasis) pathway. In some embodiments, the present disclosure provides a method for (i) predicting (e.g., generating) one or more compounds that induce a perturbation in the Chagas disease (e.g., American trypanosomiasis) pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the present disclosure provides a classifier model for predicting (e.g., generating) one or more compounds active in (e.g., induces a perturbation in) the oocyte meisosis pathway. In some embodiments, the present disclosure provides a classifier model for predicting (e.g., generating) compounds that activate and/or inhibit the oocyte meisosis pathway. In some embodiments, the present disclosure provides a method for (i) predicting (e.g., generating) one or more compounds that induce a perturbation in the oocyte meisosis pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the present disclosure provides a classifier model for predicting (e.g., generating) one or more compounds active in (e.g., induces a perturbation in) the nucleotide excision repair pathway. In some embodiments, the present disclosure provides a classifier model for predicting (e.g., generating) compounds that activate and/or inhibit the nucleotide excision repair pathway. In some embodiments, the present disclosure provides a method for (i) predicting (e.g., generating) one or more compounds that induce a perturbation in the nucleotide excision repair pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the present disclosure provides a classifier model for predicting (e.g., generating) one or more compounds active in (e.g., induces a perturbation in) the pancreatic secretion pathway. In some embodiments, the present disclosure provides a classifier model for predicting (e.g., generating) compounds that activate and/or inhibit the pancreatic secretion pathway. In some embodiments, the present disclosure provides a method for (i) predicting (e.g., generating) one or more compounds that induce a perturbation in the pancreatic secretion pathway and (ii) applying the one or more compounds to a subject (e.g., an animal or a human subject) to induce a perturbation in the respective pathway. In some embodiments, the method further comprises synthesizing the one or more compounds prior to the applying the one or more compounds to the subject.

In some embodiments, the classifier is any of the classifiers disclosed in greater detail herein (see. “Constrained Representation Learning,” above). For example, in some embodiments, the classifier is, for example, a logistic regression classifier, a k-nearest neighbor classifier, a deep neural network classifier, a support vector machine classifier, a decision tree classifier, or a naïve Bayes classifier, etc.

In some embodiments, the present disclosure further provides a method of training a classifier model for predicting (e.g., generating) one or more compounds with desired biological properties. In some embodiments, the desired biological property is an ability to induce a perturbation in a cellular and/or biological pathway. For example, in some embodiments, the cellular and/or biological pathway is a pathway involved in arachidonic acid metabolism, alpha-linolenic acid metabolism, insulin secretion, proteasome, synaptic vesicle cycle, human T-cell leukemia virus 1 infection, cytosolic DNA sensing pathway, calcium signaling pathway. Chagas disease (e.g., American trypanosomiasis), oocyte meisosis, nucleotide excision repair, and/or pancreatic secretion. In some embodiments, the cellular and/or biological pathway is a pathway selected from the KEGG pathway database, available on the Internet at www.genome.jp/kegg/pathway.html. In some embodiments, the perturbation in the respective cellular and/or biological pathway is an activation and/or an inhibition in the respective pathway. Thus, in some embodiments, the present disclosure provides a method of training a classifier model for predicting (e.g., generating) compounds capable of activating and/or inhibiting a respective cellular and/or biological pathway, such as a pathway selected from the KEGG pathway database. In some embodiments, the classifier is any of the classifiers disclosed in greater detail above, and/or any substitutions, deletions, additions, modifications, and/or combinations thereof, as will be apparent to one skilled in the art.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer readable storage medium. For instance, the computer program product could contain the program modules shown in any combination of FIG. 1 or 2. These program modules can be stored on a CD-ROM, DVD, magnetic disk storage product, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method of discovering a test compound that has a first biological property, the method comprising:
at a computer system comprising at least one processor and a memory storing at least one program for execution by the at least one processor, the at least one program comprising instructions for:
A) obtaining a first training dataset, in electronic form, wherein:
the first training dataset comprises, for each respective compound in a first plurality of compounds, (i) information regarding a chemical structure of the respective compound and (ii) one or more biological properties, in a plurality of biological properties, of the respective compound,
the first plurality of compounds comprises 100 or more compounds, and
the plurality of biological properties includes the first biological property:
B) training an untrained or partially untrained neural network encoder and an untrained or partially untrained classifier by performing a first procedure that comprises:
(i) for each respective compound in the first plurality of compounds, (a) projecting the information regarding the chemical structure of the respective compound into a latent representation space in accordance with a first plurality of weights associated with the untrained or partially untrained neural network encoder to obtain a corresponding projected representation of the respective compound, and (b) inputting the corresponding projected representation of the respective compound into the untrained or partially untrained classifier to obtain a classification of the respective compound in accordance with a second plurality of weights associated with the untrained or partially untrained classifier; and
(ii) updating the first plurality of weights and the second plurality of weights by comparing the classification of each respective compound in the first plurality of compounds to the one or more biological properties of the respective compound in the first training dataset thereby obtaining a trained neural network encoder and a trained classifier:
C) obtaining a second training dataset, in electronic form, wherein the second training dataset comprises, for each respective compound in a second plurality of compounds, information regarding a chemical structure of the respective compound and wherein the second plurality of compounds comprises 100 or more compounds;
D) training an untrained or partially untrained decoder by performing a second procedure that comprises:
(i) for each respective compound in the second plurality of compounds, (a) projecting the information regarding the chemical structure of the respective compound into a latent representation space in accordance with the first plurality of weights associated with the trained neural network encoder to obtain a corresponding projected representation of the respective compound, and (b) inputting the corresponding projected representation of the respective compound into the untrained or partially untrained decoder to obtain a chemical structure of the respective compound in accordance with a third plurality of weights associated with the untrained or partially untrained decoder; and
(ii) updating the third plurality of weights by comparing the chemical structure of each respective compound outputted by the untrained or partially untrained decoder to the actual chemical structure of the respective compound from the second training dataset thereby obtaining a trained decoder; and
E) using the trained neural network encoder, trained classifier, and trained decoder to identify a test compound that has the first biological property, wherein the test compound is not present in the first and second training set.

2. The method of claim 1, wherein the information regarding a chemical structure of the respective compound in the first plurality of compounds is a chemical structure of the respective compound or a high dimensional vector representation based upon a chemical structure of the respective compound.

3. The method of claim 1, wherein the E) using comprises:
interpolating a projected representation of a first compound and a projected representation of a second compound, produced by the trained neural network encoder, wherein the first and second compound have the first molecular property thereby obtaining an interpolated projection;
inputting the interpolated projection into the trained decoder thereby obtaining a plurality of candidate compounds;
for each respective candidate compound in all or a portion of the plurality of candidate compounds:
(i) obtaining a corresponding projected representation for the respective candidate compound by inputting a chemical structure of the candidate compound into the trained neural network encoder; and
(ii) obtaining a classification of the respective candidate compound by inputting the corresponding projected representation of the respective candidate compound into the trained classifier, wherein, when the trained classifier indicates that the corresponding projected representation of the respective candidate compound has the first biological property, the respective candidate compound is deemed to have the first biological property.

4. The method of claim 3, the method further comprising verifying a first compound in the plurality of candidate compounds has the first biological property by a third procedure that comprises:
subjecting the first compound to a wet lab assay that verifies that the respective candidate compound has the first biological property.

5. The method of claim 4, the method further comprising:
synthesizing the first compound.

6. The method of claim 1, the method further comprising verifying the trained neural network encoder, trained classifier, and trained decoder by a third procedure that comprises:
obtaining a first compound, not present in the first or second training dataset, that has the first biological property and has a known chemical structure;
obtaining a projected representation for the first compound by inputting a chemical structure of the first compound into the trained neural network encoder;
inputting the projected representation of the first compound into the trained classifier to verify that the trained classifier identifies the first compound as having the first biological property; and
inputting the projected representation of the first compound into the trained decoder to verify that the trained decoder reconstructs the chemical structure of the first compound.

7. The method of any one of claims 1-6, wherein
(i) the information regarding the chemical structure of the respective compound is a molecular structure of the respective compound,
(ii) the method further comprises:
forming a featurization of the chemical structure; and
incorporating the featurization of the chemical structure into a multi-dimensional vector space, and
(iii) the projecting the information regarding the chemical structure of the respective compound into the latent representation space in accordance with the first plurality of weights associated with the untrained or partially untrained neural network encoder comprises inputting the multi-dimensional vector space of the chemical structure into the untrained or partially untrained neural network encoder.

8. The method of claim 7, wherein the featurization of the chemical structure is a tensor.

9. The method of claim 8, wherein the tensor is a one-dimensional vector or a two-dimensional matrix.

10. The method of claim 7, wherein the featurization of the chemical structure is an extended circular fingerprint, or a molecular graph of a plurality of one-hot-encoded vectors.

11. The method of claim 7, wherein the multi-dimensional vector space is an N-dimensional space, wherein N is an integer between 20 and 80.

12. The method of claim 11, wherein N is 50.

13. The method of claim 7, wherein the incorporating the featurization of the chemical structure into the multi-dimensional vector space for the chemical structure comprises inputting the featurization of the chemical structure into a spatial graph convolutional network (GCN).

14. The method of claim 13, wherein the GCN is a graph attention network (GAT) or a graph substructure index-based approximate graph (SAGA).

15. The method of claim 7, wherein the incorporating the featurization of the molecular structure into the multi-dimensional vector space for the chemical structure comprises an application of a spectral graph convolution (SGC) to the featurization of the chemical structure.

16. The method of claim 15, wherein the application of the SGC to the featurization of the chemical structure uses Chebyshev polynomial filtering.

17. The method of claim 7, wherein the forming the featurization of the chemical structure comprises:
converting the chemical structure to a simplified molecular-input line-entry system (SMILES) string, and
converting the SMILES string into a molecular graph representation that comprises an adjacency matrix and a feature matrix.

18. The method of any one of claims 1-17, wherein the first biological property is selected from the group consisting of: an indication as to whether a compound activates a cell state, an indication as to whether a compound inhibits a cell state, an affinity for a biological target, an EC50 of the compound for inhibiting a biological state, an IC50 of the compound for inhibiting a biological state, an ED50 of the compound for inhibiting a biological state, an LD50 of the compound for inhibiting a biological state, and a TD50 of the compound for inhibiting a biological state.

19. The method of claim 18, wherein the cell state is characterized by an up-regulation or down-regulation of one or more respective genes in a plurality of genes associated with the cell state.

20. The method of claim 18, wherein the cell state is a diseased state.

21. The method of claim 18, wherein the cell state is characterized by an upregulation or a down-regulation of one or more biological pathways.

22. The method of claim 18, wherein the cell state is characterized by an upregulation or a down-regulation of one or more biological pathways in a plurality of biological pathways.

23. The method of claim 18, wherein the cell state is characterized by an upregulation or a down-regulation of one or more of cellular-components.

24. The method of claim 23, wherein the one or more cellular-components comprises a plurality of genes, optionally measured at the RNA level.

25. The method of claim 23, wherein the one or more cellular-components are quantified using single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, or any combinations thereof, or summaries of the same, including combinations, such as linear combinations, representing activated pathways in the single-cell cellular-component expression datasets.

26. The method of claim 23, wherein the one or more cellular-components comprises a plurality of proteins.

27. A computer system, comprising one or more processors and memory, the memory storing instructions for performing a method for discovering a test compound that has a first biological property, the method comprising:
A) obtaining a first training dataset, in electronic form, wherein:
the first training dataset comprises, for each respective compound in a first plurality of compounds, (i) information regarding a chemical structure of the respective compound and (ii) one or more biological properties, in a plurality of biological properties, of the respective compound,
the first plurality of compounds comprises 100 or more compounds, and
the plurality of biological properties includes the first biological property,
B) training an untrained or partially untrained neural network encoder and an untrained or partially untrained classifier by performing a first procedure that comprises:
(i) for each respective compound in the first plurality of compounds, (a) projecting the information regarding the chemical structure of the respective compound into a latent representation space in accordance with a first plurality of weights associated with the untrained or partially untrained neural network encoder to obtain a corresponding projected representation of the respective compound, and (b) inputting the corresponding projected representation of the respective compound into the untrained or partially untrained classifier to obtain a classification of the respective compound in accordance with a second plurality of weights associated with the untrained or partially untrained classifier; and
(ii) updating the first plurality of weights and the second plurality of weights by comparing the classification of each respective compound in the first plurality of compounds to the one or more biological properties of the respective compound in the first training dataset thereby obtaining a trained neural network encoder and a trained classifier;
C) obtaining a second training dataset, in electronic form, wherein the second training dataset comprises, for each respective compound in a second plurality of compounds, information regarding a chemical structure of the respective compound and wherein the second plurality of compounds comprises 100 or more compounds;
D) training an untrained or partially untrained decoder by performing a second procedure that comprises:
(i) for each respective compound in the second plurality of compounds, (a) projecting the information regarding the chemical structure of the respective compound into a latent representation space in accordance with the first plurality of weights associated with the trained neural network encoder to obtain a corresponding projected representation of the respective compound, and (b) inputting the corresponding projected representation of the respective compound into the untrained or partially untrained decoder to obtain a chemical structure of the respective compound in accordance with a third plurality of weights associated with the untrained or partially untrained decoder; and
(ii) updating the third plurality of weights by comparing the chemical structure of each respective compound outputted by the untrained or partially untrained decoder to the actual chemical structure of the respective compound from the second training dataset thereby obtaining a trained decoder; and
E) using the trained neural network encoder, trained classifier, and trained decoder to identify a test compound that has the first biological property, wherein the test compound is not present in the first and second training set.

28. A non-transitory computer-readable medium storing one or more computer programs, executable by a computer, for performing a method for discovering a test compound that has a first biological property, the computer comprising one or more processors and a memory, the one or more computer programs collectively encoding computer executable instructions that perform a method comprising:
A) obtaining a first training dataset, in electronic form, wherein:
the first training dataset comprises, for each respective compound in a first plurality of compounds, (i) information regarding a chemical structure of the respective compound and (ii) one or more biological properties, in a plurality of biological properties, of the respective compound,
the first plurality of compounds comprises 100 or more compounds, and
the plurality of biological properties includes the first biological property;
B) training an untrained or partially untrained neural network encoder and an untrained or partially untrained classifier by performing a first procedure that comprises:
(i) for each respective compound in the first plurality of compounds, (a) projecting the information regarding the chemical structure of the respective compound into a latent representation space in accordance with a first plurality of weights associated with the untrained or partially untrained neural network encoder to obtain a corresponding projected representation of the respective compound, and (b) inputting the corresponding projected representation of the respective compound into the untrained or partially untrained classifier to obtain a classification of the respective compound in accordance with a second plurality of weights associated with the untrained or partially untrained classifier; and
(ii) updating the first plurality of weights and the second plurality of weights by comparing the classification of each respective compound in the first plurality of compounds to the one or more biological properties of the respective compound in the first training dataset thereby obtaining a trained neural network encoder and a trained classifier;
C) obtaining a second training dataset, in electronic form, wherein the second training dataset comprises, for each respective compound in a second plurality of compounds, information regarding a chemical structure of the respective compound and wherein the second plurality of compounds comprises 100 or more compounds;
D) training an untrained or partially untrained decoder by performing a second procedure that comprises:
(i) for each respective compound in the second plurality of compounds, (a) projecting the information regarding the chemical structure of the respective compound into a latent representation space in accordance with the first plurality of weights associated with the trained neural network encoder to obtain a corresponding projected representation of the respective compound, and (b) inputting the corresponding projected representation of the respective compound into the untrained or partially untrained decoder to obtain a chemical structure of the respective compound in accordance with a third plurality of weights associated with the untrained or partially untrained decoder; and
(ii) updating the third plurality of weights by comparing the chemical structure of each respective compound outputted by the untrained or partially untrained decoder to the actual chemical structure of the respective compound from the second training dataset thereby obtaining a trained decoder; and
E) using the trained neural network encoder, trained classifier, and trained decoder to identify a test compound that has the first biological property, wherein the test compound is not present in the first and second training set.

29. A method of discovering a candidate compound that has a first biological property, the method comprising:
at a computer system comprising at least one processor and a memory storing at least one program for execution by the at least one processor, the at least one program comprising instructions for:
obtaining a first projected representation of a first compound that is assigned the first biological property by inputting a chemical structure of the first compound into a trained neural network encoder, wherein the first projected representation has N dimensions, wherein N is an integer between 20 and 80;
using the first projection to obtain one or more candidate projections;
inputting each candidate projection in the one or more candidate projections into a trained decoder thereby obtaining a plurality of candidate compounds, wherein the first compound is not present in the plurality of candidate compounds;
for each respective candidate compound in the plurality of candidate compounds:
(i) obtaining a corresponding projected representation for the respective candidate compound by inputting a chemical structure of the candidate compound into the trained neural network encoder, wherein the corresponding projected representation has N dimensions; and
(ii) obtaining a classification of the respective candidate compound by inputting the corresponding projected representation of the respective candidate compound into the trained classifier, wherein, w % ben the trained classifier indicates that the corresponding projected representation of the respective candidate compound has the first biological property, the respective candidate compound is deemed to have the first biological property.

30. The method of claim 29, the method further comprising:
obtaining a second projected representation of a second compound that has the biological property by inputting a chemical structure of the second compound into the trained neural network encoder, and wherein
the using the first projection to obtain one or more candidate projections comprises interpolating the first projection and the second projection thereby obtaining the one or more candidate projections.

31. A computer system, comprising one or more processors and memory, the memory storing instructions for performing a method of discovering a candidate compound that has a first biological property, the method comprising:
obtaining a first projected representation of a first compound that is assigned the first biological property by inputting a chemical structure of the first compound into a trained neural network encoder, wherein the first projected representation has N dimensions, wherein N is an integer between 20 and 80;
using the first projection to obtain one or more candidate projections:
inputting each candidate projection in the one or more candidate projections into the trained decoder thereby obtaining a plurality of candidate compounds, wherein the first compound is not present in the plurality of candidate compounds;
for each respective candidate compound in the plurality of candidate compounds:
(i) obtaining a corresponding projected representation for the respective candidate compound by inputting a chemical structure of the candidate compound into the trained neural network encoder, wherein the corresponding projected representation has N dimensions; and
(ii) obtaining a classification of the respective candidate compound by inputting the corresponding projected representation of the respective candidate compound into the trained classifier, wherein, when the trained classifier indicates that the corresponding projected representation of the respective candidate compound has the first biological property, the respective candidate compound is deemed to have the first biological property.

32. A non-transitory computer-readable medium storing one or more computer programs, executable by a computer, for performing a method of discovering a candidate compound that has a first biological property, the computer comprising one or more processors and a memory, the one or more computer programs collectively encoding computer executable instructions that perform a method comprising:
obtaining a first projected representation of a first compound that is assigned the first biological property by inputting a chemical structure of the first compound into a trained neural network encoder, wherein the first projected representation has N dimensions, wherein N is an integer between 20 and 80;
using the first projection to obtain one or more candidate projections;
inputting each candidate projection in the one or more candidate projections into the trained decoder thereby obtaining a plurality of candidate compounds, wherein the first compound is not present in the plurality of candidate compounds;
for each respective candidate compound in the plurality of candidate compounds:
(i) obtaining a corresponding projected representation for the respective candidate compound by inputting a chemical structure of the candidate compound into the trained neural network encoder, wherein the corresponding projected representation has N dimensions, and
(ii) obtaining a classification of the respective candidate compound by inputting the corresponding projected representation of the respective candidate compound into the trained classifier, wherein, when the trained classifier indicates that the corresponding projected representation of the respective candidate compound has the first biological property, the respective candidate compound is deemed to have the first biological property.

33. The method of claim 29, wherein the first biological property is a compound function.

34. The method of claim 29, further comprising:
subjecting the respective candidate compound to a wet lab assay that verifies that the respective candidate compound has the first biological property.

35. The method of claim 34, the method further comprising synthesizing the respective candidate compound.

36. A method of discovering a test compound that has a first biological property, the method comprising:
at a computer system comprising at least one processor and a memory storing at least one program for execution by the at least one processor, the at least one program comprising instructions for:
using a trained neural network encoder, trained classifier, and trained decoder to identify a test compound that has a first biological property, wherein the trained neural network encoder, trained classifier, and trained decoder were trained by processes comprising:
A) obtaining a first training dataset, in electronic form, wherein:
the first training dataset comprises, for each respective compound in a first plurality of compounds, (i) information regarding a chemical structure of the respective compound and (ii) one or more biological properties, in a plurality of biological properties, of the respective compound,
the first plurality of compounds comprises 100 or more compounds, and
the plurality of biological properties includes the first biological property;
B) training an untrained or partially untrained neural network encoder and an untrained or partially untrained classifier by performing a first procedure that comprises:
(i) for each respective compound in the first plurality of compounds, (a) projecting the information regarding the chemical structure of the respective compound into a latent representation space in accordance with a first plurality of weights associated with the untrained or partially untrained neural network encoder to obtain a corresponding projected representation of the respective compound, and (b) inputting the corresponding projected representation of the respective compound into the untrained or partially untrained classifier to obtain a classification of the respective compound in accordance with a second plurality of weights associated with the untrained or partially untrained classifier; and
(ii) updating the first plurality of weights and the second plurality of weights by comparing the classification of each respective compound in the first plurality of compounds to the one or more biological properties of the respective compound in the first training dataset thereby obtaining a trained neural network encoder and a trained classifier;
C) obtaining a second training dataset, in electronic form, wherein the second training dataset comprises, for each respective compound in a second plurality of compounds, information regarding a chemical structure of the respective compound, and wherein the second plurality of compounds comprises 100 or more compounds; and
D) training an untrained or partially untrained decoder by performing a second procedure that comprises:
(i) for each respective compound in the second plurality of compounds, (a) projecting the information regarding the chemical structure of the respective compound into a latent representation space in accordance with the first plurality of weights associated with the trained neural network encoder to obtain a corresponding projected representation of the respective compound, and (b) inputting the corresponding projected representation of the respective compound into the untrained or partially untrained decoder to obtain a chemical structure of the respective compound in accordance with a third plurality of weights associated with the untrained or partially untrained decoder; and
(ii) updating the third plurality of weights by comparing the chemical structure of each respective compound outputted by the untrained or partially untrained decoder to the actual chemical structure of the respective compound from the second training dataset thereby obtaining a trained decoder;
wherein the test compound is not present in the first and second training set.

37. A computer system, comprising one or more processors and memory, the memory storing instructions for performing a method for discovering a test compound that has a first biological property, the method comprising:
using a trained neural network encoder, trained classifier, and trained decoder to identify a test compound that has the first biological property, wherein the trained neural network encoder, trained classifier, and trained decoder were trained by processes comprising:
A) obtaining a first training dataset, in electronic form, wherein:
the first training dataset comprises, for each respective compound in a first plurality of compounds, (i) information regarding a chemical structure of the respective compound and (ii) one or more biological properties, in a plurality of biological properties, of the respective compound,
the first plurality of compounds comprises 100 or more compounds, and
the plurality of biological properties includes the first biological property;
B) training an untrained or partially untrained neural network encoder and an untrained or partially untrained classifier by performing a first procedure that comprises:
(i) for each respective compound in the first plurality of compounds, (a) projecting the information regarding the chemical structure of the respective compound into a latent representation space in accordance with a first plurality of weights associated with the untrained or partially untrained neural network encoder to obtain a corresponding projected representation of the respective compound, and (b) inputting the corresponding projected representation of the respective compound into the untrained or partially untrained classifier to obtain a classification of the respective compound in accordance with a second plurality of weights associated with the untrained or partially untrained classifier; and
(ii) updating the first plurality of weights and the second plurality of weights by comparing the classification of each respective compound in the first plurality of compounds to the one or more biological properties of the respective compound in the first training dataset thereby obtaining a trained neural network encoder and a trained classifier;
C) obtaining a second training dataset, in electronic form, wherein the second training dataset comprises, for each respective compound in a second plurality of compounds, information regarding a chemical structure of the respective compound, and wherein the second plurality of compounds comprises 100 or more compounds; and
D) training an untrained or partially untrained decoder by performing a second procedure that comprises:
(i) for each respective compound in the second plurality of compounds, (a) projecting the information regarding the chemical structure of the respective compound into a latent representation space in accordance with the first plurality of weights associated with the trained neural network encoder to obtain a corresponding projected representation of the respective compound, and (b) inputting the corresponding projected representation of the respective compound into the untrained or partially untrained decoder to obtain a chemical structure of the respective compound in accordance with a third plurality of weights associated with the untrained or partially untrained decoder; and
(ii) updating the third plurality of weights by comparing the chemical structure of each respective compound outputted by the untrained or partially untrained decoder to the actual chemical structure of the respective compound from the second training dataset thereby obtaining a trained decoder,
wherein the test compound is not present in the first and second training set.

38. A non-transitory computer-readable medium storing one or more computer programs, executable by a computer, for performing a method for discovering a test compound that has a first biological property, the computer comprising one or more processors and a memory, the one or more computer programs collectively encoding computer executable instructions that perform a method comprising: using a trained neural network encoder, trained classifier, and trained decoder to identify a test compound that has the first biological property, wherein the trained neural network encoder, trained classifier, and trained decoder were trained by processes comprising:
A) obtaining a first training dataset, in electronic form, wherein:
the first training dataset comprises, for each respective compound in a first plurality of compounds, (i) information regarding a chemical structure of the respective compound and (ii) one or more biological properties, in a plurality of biological properties, of the respective compound
the first plurality of compounds comprises 100 or more compounds, and
the plurality of biological properties includes the first biological property;
B) training an untrained or partially untrained neural network encoder and an untrained or partially untrained classifier by performing a first procedure that comprises:
(i) for each respective compound in the first plurality of compounds, (a) projecting the information regarding the chemical structure of the respective compound into a latent representation space in accordance with a first plurality of weights associated with the untrained or partially untrained neural network encoder to obtain a corresponding projected representation of the respective compound, and (b) inputting the corresponding projected representation of the respective compound into the untrained or partially untrained classifier to obtain a classification of the respective compound in accordance with a second plurality of weights associated with the untrained or partially untrained classifier; and
(ii) updating the first plurality of weights and the second plurality of weights by comparing the classification of each respective compound in the first plurality of compounds to the one or more biological properties of the respective compound in the first training dataset thereby obtaining a trained neural network encoder and a trained classifier;
C) obtaining a second training dataset, in electronic form, wherein the second training dataset comprises, for each respective compound in a second plurality of compounds, information regarding a chemical structure of the respective compound, and wherein the second plurality of compounds comprises 100 or more compounds; and
D) training an untrained or partially untrained decoder by performing a second procedure that comprises:
(i) for each respective compound in the second plurality of compounds, (a) projecting the information regarding the chemical structure of the respective compound into a latent representation space in accordance with the first plurality of weights associated with the trained neural network encoder to obtain a corresponding projected representation of the respective compound, and (b) inputting the corresponding projected representation of the respective compound into the untrained or partially untrained decoder to obtain a chemical structure of the respective compound in accordance with a third plurality of weights associated with the untrained or partially untrained decoder; and
(ii) updating the third plurality of weights by comparing the chemical structure of each respective compound outputted by the untrained or partially untrained decoder to the actual chemical structure of the respective compound from the second training dataset thereby obtaining a trained decoder;
wherein the test compound is not present in the first and second training set.

39. A method of synthesizing a test compound that has a first biological property, wherein the compound was designed by a method comprising:
at a computer system comprising at least one processor and a memory storing at least one program for execution by the at least one processor, the at least one program comprising instructions for:
A) obtaining a first training dataset, in electronic form, wherein:
the first training dataset comprises, for each respective compound in a first plurality of compounds, (i) information regarding a chemical structure of the respective compound and (ii) one or more biological properties, in a plurality of biological properties, of the respective compound
the first plurality of compounds comprises 100 or more compounds, and
the plurality of biological properties includes the first biological property;
B) training an untrained or partially untrained neural network encoder and an untrained or partially untrained classifier by performing a first procedure that comprises:
(i) for each respective compound in the first plurality of compounds, (a) projecting the information regarding the chemical structure of the respective compound into a latent representation space in accordance with a first plurality of weights associated with the untrained or partially untrained neural network encoder to obtain a corresponding projected representation of the respective compound, and (b) inputting the corresponding projected representation of the respective compound into the untrained or partially untrained classifier to obtain a classification of the respective compound in accordance with a second plurality of weights associated with the untrained or partially untrained classifier; and
(ii) updating the first plurality of weights and the second plurality of weights by comparing the classification of each respective compound in the first plurality of compounds to the one or more biological properties of the respective compound in the first training dataset thereby obtaining a trained neural network encoder and a trained classifier:
C) obtaining a second training dataset, in electronic form, wherein the second training dataset comprises, for each respective compound in a second plurality of compounds, information regarding a chemical structure of the respective compound, and wherein the second plurality of compounds comprises 100 or more compounds;
D) training an untrained or partially untrained decoder by performing a second procedure that comprises:
(i) for each respective compound in the second plurality of compounds, (a) projecting the information regarding the chemical structure of the respective compound into a latent representation space in accordance with the first plurality of weights associated with the trained neural network encoder to obtain a corresponding projected representation of the respective compound, and (b) inputting the corresponding projected representation of the respective compound into the untrained or partially untrained decoder to obtain a chemical structure of the respective compound in accordance with a third plurality of weights associated with the untrained or partially untrained decoder; and
(ii) updating the third plurality of weights by comparing the chemical structure of each respective compound outputted by the untrained or partially untrained decoder to the actual chemical structure of the respective compound from the second training dataset thereby obtaining a trained decoder; and
E) using the trained neural network encoder, trained classifier, and trained decoder to identify the test compound that has the first biological property, wherein the test compound is not present in the first and second training set.
